(19) United States
(12) Patent Application Publication
(10) Pub. No.: US 2020/0060731 A1
(43) Pub. Date: Feb. 27, 2020

(54) Advanced Polyaxial System and Surgical Procedure

(71) Applicants: Marc Evan Richelsoph, Belmont, NC (US); David Frederick Waller, Atlanta, GA (US)

(72) Inventors: Marc Evan Richelsoph, Belmont, NC (US); David Frederick Waller, Atlanta, GA (US)

(21) Appl. No.: 16/612,672

(22) PCT Filed: May 11, 2018
(86) PCT No.: PCT/US18/32232

§ 371 Date: Nov. 11, 2019

Related U.S. Application Data
(60) Provisional application No. 62/504,738, filed on May 11, 2017.
(60) Provisional application No. 62/569,724, filed on Oct. 9, 2017.
(60) Provisional application No. 62/653,601, filed on Apr. 6, 2018.

(51) Int. Cl. A61B 17/70; A61B 17/86

(52) U.S. Cl. CPC A61B 17/7014; A61B 17/7005; A61B 17/7037; A61B 2017/681; A61B 17/7041; A61B 17/863; A61B 17/704

(57) ABSTRACT

A spinal implant including a body having an insert received within a bottom portion of the body, the insert receiving a bone screw at a bottom portion for attachment to a spine, a rod assembly received within a rod slot on a side portion of the body wherein the rod slot does not extend through a top surface of the body, and a locking nut received within a top portion of the body and interlocking with a top portion of the insert. Spinal implant assembly, including multiple spinal implants interconnected through rod assemblies. Method of using a spinal implant by inserting spinal implant at bone structure of a spine, tightening the locking nut, drawing the body upwards, compressing the insert against the bone screw head, and contacting a lower surface of locking nut and rod assembly and compressing rod assembly against the insert, thus locking rod assembly and bone screw.

### BACKGROUND OF THE INVENTION

### 1. Technical Field

The present invention relates to spinal implant systems and surgical procedures for insertion of spinal implants.

### 2. Background Art

The insertion of pedicle screws into the spine for fixation has been commonly used for many years. In general, a set of implants is placed on both sides of the spinous process into the pedicles and the set is connected by an individual rod. For example, in a single level fusion, whereby two vertebral bodies are intended to be fused together, four pedicle screws are used, two on each side of the spinous process. Each set of two is then connected by the rod. For multiple levels, more screws are used and connected by longer rods. The general technique is an open procedure, whereby the incision in the skin is long and spans the length of the affected area of the spine to be treated. Minimally invasive techniques allow for the placement of the implants through small openings or cannula to reduce skin and muscle trauma. However, it is often challenging and tedious to introduce long rods through small openings or cannula, creating limitations on what procedures can be done this way.

The lumbar spine consists of multiple vertebrae that in a healthy spine are flexibly held within a general S curve. Each vertebrae is a different size and different geometry. The pedicles on each vertebrae, which are posts that extend from the vertebral body, vary in angle and distance apart from one vertebral body to the next. Therefore, each polyaxial screw sits at a different angle and height when inserted to the same depth into the pedicles.

Polyaxial screw technology has been in existence for a number of years. While the technology has advanced, the focus of key advances have been on providing smaller and alternate means for fixing a screw that fixes a body member and rod assembly to vertebrae, with each screw assembly having the basic structure of a body with pivot means around the screw head and a rod slot. If the rod to be disposed in the rod slot is not centered relative to the screw head, the body can pivot over to adjust for the misalignment. Examples of such systems are abundant in the art.

Once the polyaxial screw is connected to the rod, the assembly is locked such that the screw angulation is fixed relative to the body portion. For example, U.S. Pat. No. 6,740,086 to Richelsoph, issued May 25, 2004, shows one such system.

While a rod can be contoured or bent to meet the openings or saddles in the polyaxial screw, this is extremely difficult to do well. If the rod is not bent perfectly, one or more polyaxial screw saddles or openings will be below the rod in a multilevel construct. Securing the rod by normal set screws or other locking means will pull the polyaxial screw up to meet the rod, either distorting the spinal anatomy or weakening the bone interface. In certain situations, such as scoliosis, this can be a benefit to aid in realigning the spine. However, in general, it is preferred to have an ideal fit.

In addition, a key element of spinal surgery is the need for compression or distraction. This allows a surgeon to compress vertebral bodies against an interbody, bone graft, or distract to allow an interbody or bone graft into the intervertebral space or restore proper disc height to remove pressure off of nerves. Large compressor and distractor instruments are bulky, and do not provide accurate compression and distraction or tactile feedback. While a unique compressor and distractor instrument for a polyaxial plate based system is described in Richelsoph et al., U.S. Pat. Nos. 9,044,273 and 9,526,531, for a polyaxial screw system, a different and novel approach is needed to allow for a reduced polyaxial body size.

Furthermore, polyaxial screws and rod constructs, torque and counter torque are generally applied to lock the construct. To do so requires a bulky counter torque instrument that slides over the head of the polyaxial screw. This increases the bulk of the screw head and can make it more challenging to lock. In addition, the bulk also increases the size of the tube for minimally invasive access.

U.S. Pat. No. 8,535,352 to Altarac, et al. discloses a spinal alignment system for interconnecting vertebral bodies that includes a bone screw polyaxially connected to a seat, the seat including a top opening, a first rod receiving portion and a second rod receiving portion, a first rod channel and a second rod channel. The system is implanted into a first vertebral body. A first rod is introduced to the seat through the top opening in a first orientation and connected to the first rod receiving portion. A second rod is introduced to the seat through the top opening in a first orientation and connected to the second rod receiving portion. The first and second rods are each moved into a second orientation such that the rods project through the first and second rod channels, respectively. The design in Altarac, et al. is very inefficient energy-wise due to the top opening of the seat. When the rods are inserted and any set screw is tightened, this will splay the arms of the seat. Altarac tries to compensate by using a set screw and bayonet concept, but this does not eliminate splaying and reduces the amount of contact area of the rods the set screw can apply load to. This will compromise the integrity of the implant body and not provide sufficient locking. The design of Altarac, et al. further cannot work with compression and distraction procedures that treat more than two levels of the spine, or easily change out rods or implants in these two level procedures.

Therefore, while these prior polyaxial screw systems and surgical procedures can be suitable for limited usage to which they somewhat address, they are not suitable to providing a polyaxial screw based implant and surgical approach that can accurately and securely connect multiple screws together, adjust for anatomical variations, provide a low profile system, and significantly reduce the quantity of implant and instruments needed while reducing surgical complexity.

Thus, a need exists to overcome the problems with the prior art systems, designs, and processes as discussed above.

### SUMMARY OF THE INVENTION

The present invention provides for a spinal implant including a body having an insert received within a bottom portion of the body, the insert receiving a bone screw at a bottom portion for attachment to a spine, a rod assembly received within a rod slot on a side portion of the body wherein the rod slot does not extend through a top surface of the body, and a locking nut received within a top portion of the body and interlocking with a top portion of the insert.

The present invention also provides for a spinal implant assembly, including multiple spinal implants interconnected through rod assemblies.

The present invention provides for a spinal implant, including a body having an insert received within a bottom portion of the body, the insert receiving a bone screw at a bottom portion for attachment to a spine, a rod received within at least one seat on a side portion of the insert wherein the seat does not extend through a top surface of the body, the seat including at least one bearing that receives the rod, and a locking nut received within a top portion of the body and interlocking with a top portion of the insert through a snap ring.

The present invention also provides for a spinal implant including a body including at least one seat for accepting at least one pivotable bearing for receiving at least one rod, the body including a bottom portion for engaging a bone screw, and a locking nut received within a top portion of the body and engaging threads on a top portion of the bone screw such that when the locking nut is tightened, the body compresses and locks a position of the bearings.

The present invention provides for a spinal implant including a partially flexible body with an opening for receiving a bone screw, a receiver for accepting a rod that pivots independently of the body, and a locking mechanism that compresses the partially flexible body against the at least one receiver to lock the assembly.

The present invention provides for a method of using the spinal implant by inserting the spinal implant at a bone structure of a spine, tightening the locking nut, drawing the body upwards, compressing the insert against the bone screw head, and contacting a lower surface of the locking nut and the rod assembly and compressing the rod assembly against the insert, thus locking the rod assembly and bone screw.

The present invention also provides for a method of using the spinal implant by inserting a spinal implant at a bone structure of a spine including a body including two seats having pivotable bearings, receiving at least one rod into at least one pivotable bearing, a body including a bottom portion for engaging a bone screw, and a locking nut received within a top portion of the body and engaging threads on a top portion of the bone screw, tightening the locking nut, compressing the body and thereby exerting force against the bearings, and locking the position of the bearings.

The present invention also provides for a method of aligning and locking a spinal implant, by turning a locking nut, pulling a body of the spinal implant upwards and aligning an insert within the body, and locking the spinal implant.

The present invention provides for a spinal implant assembly including a first spinal implant having at least one side opening and at least one bearing for receiving a spinal rod to a specific or maximum depth, a second spinal implant having at least one side opening and at least one bearing for receiving a spinal rod to a specific or maximum depth, and a rod that can be inserted through the side opening and bearing of the first spinal implant and connected to the second spinal implant by inserting the rod through the side opening and into the bearing of the second spinal implant.

The present invention also provides for a spinal implant assembly including a first spinal implant having two bearings for receiving spinal rods and a second spinal implant having at least one bearing for receiving a spinal rod such that when the first spinal implant and the second spinal implant are implanted, at least one bearing remains open for adding on an additional rod and spinal implant.

### DESCRIPTION OF THE DRAWINGS

Other advantages of the present invention are readily appreciated as the same becomes better understood by reference to the following detailed description when considered in connection with the accompanying drawings wherein:

FIG. 1 is a view of a two polyaxial screw assembly;

FIG. 2 is a view of a single polyaxial screw assembly;

FIG. 3 is an exploded view of a polyaxial body assembly with compression distraction rod assembly;

FIG. 4 is a side view of an insert;

FIG. 5 is an iso view of the insert;

FIG. 6 is an bottom iso view of the insert;

FIG. 7 is a top iso view of a body;

FIG. 8 is a side view of a body;

FIG. 9 is a section view of a body;

FIG. 10 is an exploded view of the compression distraction rod assembly;

FIG. 11 is a view of the assembled compression distraction rod assembly;

FIG. 12 is a side view of multiple compression distraction rod assemblies on a curved rod;

FIG. 13 is a top iso view of a lock nut;

FIG. 14 is a section view of a lock nut;

FIG. 15 is a top iso view of a lock nut variation;

FIG. 16 is a top iso view of a lock nut variation;

FIG. 17 is an iso view of a snap ring;

FIG. 18 is a cutaway view of a lock nut showing the snap ring and insert;

FIG. 19 is an iso view of a polyaxial assembly with the insert in position to accept a bone screw;

FIG. 20 is a side view of the polyaxial assembly with the insert in position to accept a bone screw;

FIG. 21 is an iso top view of a polyaxial screw assembly and compression distraction instrument;

FIG. 22 is an side view of a polyaxial screw assembly and compression distraction instrument;

FIG. 23 is an iso view of the compression distraction instrument;

FIG. 24 is a side view of two polyaxial screw assemblies on a compression distraction rod assembly;

FIG. 25 is an exploded view of a polyaxial assembly variation;

FIG. 26 is a section view of an insert variation;

FIG. 27 is an iso view of a compression distraction assembly with spherical end;

FIG. 28 is an iso view of a polyaxial assembly, compression distraction assembly with spherical end, and insert variation;

FIG. 29 is a side view of connected multiple polyaxial screw bodies;

FIG. 30 is a top iso view of an compression distraction assembly and body variation;

FIG. 31 is a top iso view of a compression distraction assembly variation;

FIG. 32 is a side iso view of an assembly variation;

FIG. 33 is an iso view of a compression distraction rod assembly variation;

FIG. 34 is a top iso view of a dual level construct variation partially engaged;

FIG. 35 is a top iso view of a dual level construct variation fully engaged;

FIG. 36 is a side view of a polyaxial screw assembly and compression distraction variation for location on the multi-positional rod;

FIG. 37 is an iso view of a multi-positional rod;

FIG. 38 is a side view of two polyaxial screw assemblies on a rod;

FIG. 39 is an iso top view of a multilevel polyaxial screw assembly;

FIG. 40 is a close up view of a polyaxial body assembly and rack engaged with an instrument;

FIG. 41 is an iso view of a polyaxial screw assembly containing at least one bearing;

FIG. 42 is an exploded assembly view of polyaxial screw assembly containing two bearings;

FIG. 43 is an iso view of a polyaxial insert and two bearings;

FIG. 44 is a section view of a polyaxial insert and two bearings;

FIG. 45 is an iso view of a polyaxial body variation;

FIG. 46 is an iso view of a rod example;

FIG. 47 is a top view of multiple polyaxial assemblies with bearings connected by rods;

FIG. 48 is an iso view of multiple polyaxial assemblies with bearings connected by rods;

FIG. 49 is an exploded view of the polyaxial screw assembly with an insert in two pieces;

FIG. 50 is a side view of a lower section of an insert;

FIG. 51 is a side view of an upper section of an insert;

FIG. 52 is a side view of upper and lower inserts assembled with bearings;

FIG. 53 is a side view of a variation of a body;

FIG. 54 is a side view of a variation of a body with a key shaped rod slot;

FIG. 55 is a section view of an assembly in a locked position;

FIG. 56 is a top section view of the assembly;

FIG. 57 is a side view of a polyaxial screw assembly;

FIG. 58 is an exploded view of a polyaxial screw assembly;

FIG. 59 is a side view of a bearing;

FIG. 60 is a side view of a bone screw;

FIG. 61 is an exploded view of a body assembly;

FIG. 62 is a side view of the body assembly;

FIG. 63 is a section view of a body;

FIG. 64 is a side view of a locknut;

FIG. 65 is a top view of an assembly engaged on bone screws;

FIG. 66 is a top view of an assembly engaged on bone screws;

FIG. 67 is a top view of three assemblies connected by rods;

FIG. 68 is a top view of the three assemblies with straightened rods;

FIG. 69 is an exploded view of an assembly and external clip;

FIG. 70 is a side view of an assembly and external clip;

FIG. 71 is a side view of an instrumentation approach in the assembly;

FIG. 72 is a close-up side view of an instrumentation approach in the assembly;

FIG. 73 is a side view of an assembly with a bearing;

FIG. 74 is a side view of a rod; and

FIG. 75 is a side view of a body.

### DETAILED DESCRIPTION OF THE INVENTION

The invention provides a new implant system for adjusting to the anatomy of the spine and connecting two or more vertebral bodies securely that overcomes the mentioned disadvantages of the heretofore-known devices and methods of this general type and that provide such features by substantially departing from the conventional concepts and designs of the prior art, and in so doing allow simpler and more accurate connection of multiple spinal implants while providing a small overall size leading to less trauma to soft tissue. Most generally, the present invention provides for a spinal implant including a body having an insert received within a bottom portion of the body, the insert receiving a bone screw at a bottom portion for attachment to a spine, a rod assembly received within a rod slot on a side portion of the body wherein the rod slot does not extend through a top surface of the body, and a locking nut received within a top portion of the body and interlocking with a top portion of the insert. The present invention also provides for a spinal implant assembly, including multiple spinal implants interconnected through rod assemblies, further described below. One of the advantages of the present invention is that with a body that is not split or extend through a top surface of the body (i.e., open at a top side to receive a rod) as in the prior art but rather the body maintains a solid ring of material above the rod slot, no part of the body splays, and locking the implant in place is energy efficient. Therefore, the present invention generally provides for a spinal implant, including a partially flexible body with an opening for receiving a bone screw, a receiver for accepting a rod that pivots independently of the body, and a locking mechanism that compresses the partially flexible body against the at least one receiver to lock the assembly.

Before the present invention is disclosed and described, it is to be understood that the terminology used herein is for the purpose of describing particular embodiments only and is not intended to be limiting. The terms “a” or “an”, as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

Relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

Herein various embodiments of the present invention are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

Described now are exemplary embodiments of the present invention. Referring now to the figures of the drawings in detail, there is shown a first exemplary embodiment of a new implant, illustrated generally at 100, that, by its novel construction, permits the implant 100 to attach to bone screws while compensating for anatomic considerations and allowing for accurate compression and distraction of the spine by a rod assembly having a component having unique features.

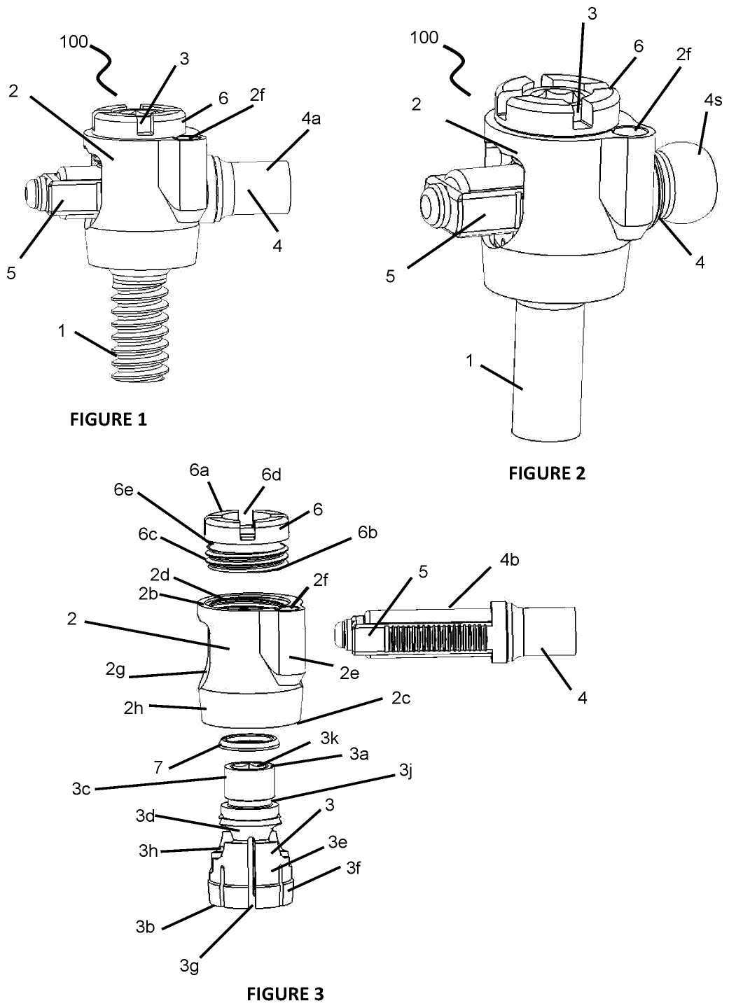

As shown in FIG. 1, the embodiment 100 of a new implant consists of a polyaxial screw having a spinal implant, generally a bone screw 1, for insertion into or attachment to the spine, a body 2, and insert 3, a rod or rod portion 4 having a rack member 5, and a locking nut/screw 6. These elements will be broken down into greater detail in the following figures. The rod portion 4 shown here ends in a cylindrical rod 4a. It is obvious to one skilled in that art that the implant is designed to be connected to other implants and there are different ways to accomplish this. The bone screw 1 can be a polyaxial screw or a monoaxial screw, a single lead threaded bone screw, or multiple lead threaded bone screws.

As shown in FIG. 2, the implant 100 has a rod assembly that ends in a spherical shape 4s. This shape can be any shape that connects with another implant. This will be further detailed in the following text and figures.

FIG. 3 shows an exploded view of the components that make up an assembly of embodiment 100. The body 1 is substantially round in shape. Of course, the body 1 does not need to be round, but can also be other shapes, and can be cannulated, or have a textured spherical or semi-spherical surface. The body 2 has an upper surface 2b and a lower surface 2c. A threaded section 2d extends partially downwards from the upper surface 2b. An extension 2e from body 1 has an opening or bore 2f extending downwards from the upper surface 2b. An opening 2g on either side of the body allows rod assembly 5 to enter. The lower section 2h of body 1 can be tapered or chamfered. The insert 3 has a top surface 3a, bottom surface 3b, top cylindrical portion 3c, a reduced diameter section 3d, a section 3e, preferably cylindrical but can be tapered or another shape, a tapered section 3f, at least one slot 3g, an opening 3h through insert 3, a groove 3j, and a feature 3k, such as a hex, torx, or other feature for accepting an instrument tip. The rod 4 has an external rotating or pivoting component 5 to make a compression distraction rod assembly. A locking nut 6 consists of a top surface 6a, lower surface 6b, threads 6c extending upwards from the lower surface 6b, a feature 6d for attaching to a driving instrument, and a thread relief groove 6e to allow the nut to thread deeper into body threads 2d. A snap ring 7 fits into groove 3j in the insert 3. All of the components above will be explained in more detail in additional figures.

The body, as well as the other components of the implant can be made of various materials, such as, but not limited to, metals, such as titanium, or stainless steels, polymers, or a combination of both. All components can be machined on standard CNC equipment.

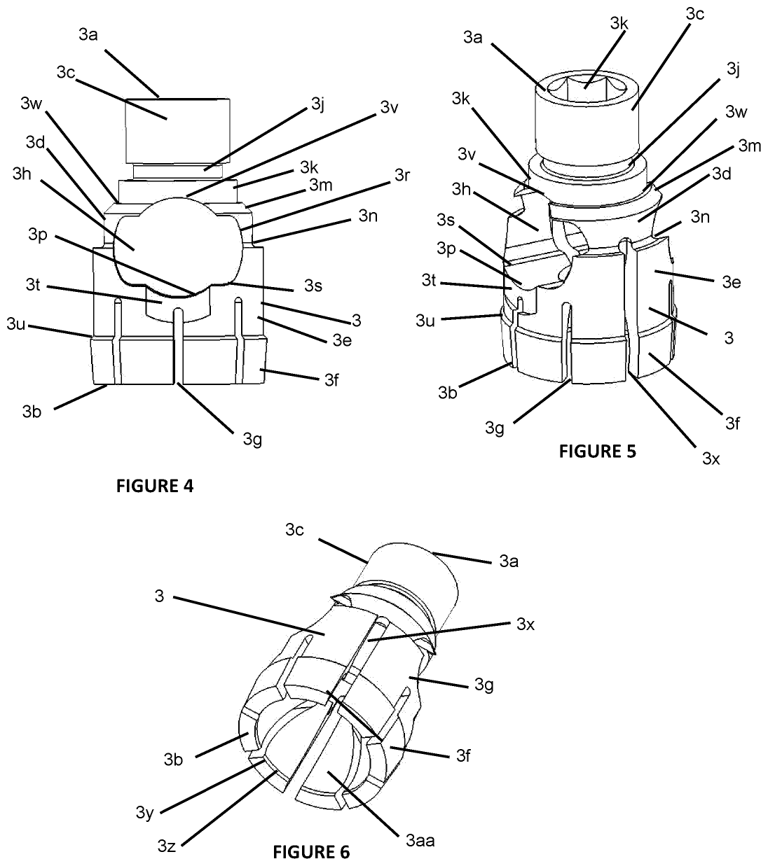

As shown in FIG. 4, the insert 3 has additional features that can be seen in the side view. The opening 3h consists of multiple features that allow for the rod assembly to enter and slide within. These include opening sides 3r that generally match the shape of the outside of rack member 5. Blend radii 3S at the four corners reduce or prevent stress risers. In addition, a rod seat 3p is present to allow a cylindrical rod portion 4b of the rod 4 to sit within. Of course, a normal spinal rod will also fit within seat 3p. The top of the rod seat 3v can be matched to the same diameter as the rod or rod portion 4b. However, as the rod assembly or rod must slide within the rod seat, it is preferred that clearance be provided and at least one of 3p or 3v or both be larger in dimensions than the rod or rod section. The reduced section 3d consists of a shoulder 3k and a chamfer 3m between the two. A blend radii 3n and 3w reduce stress risers between the sections. While shoulder 3k is larger than 3c in the figure, and it is preferred to maximize material conditions, it is not necessary if the rod assembly is reduced in size sufficiently to reduce opening 3h in the insert. The insert can have material removed or flats cut 3t to provide additional clearance that would allow the rod assembly 4 and 5 more range of motion.

In FIG. 5, the isometric view helps to clarify features and their relative positions. Note that the slots through the tapered section 3f can be of equal or different heights, however, it is preferred that slots 3x perpendicular to the rod opening be longer than the other slots. This allows for the insert to gain flexibility at the bottom 3b. The longer these slots, the more flexible the opening becomes.

In FIG. 6, the insert 3 is shown angled to detail the inner details of the lower opening 3y. Extending from the bottom 3b, there is an opening 3y. This leads to a spherical or semi-spherical shape formed or machined within insert 3. The sphere 3aa, when machined from the bottom face 3b would leave a sharp edge at the intersection with the bottom face 3b. It is preferred to chamfer this edge 3z, or create a cylindrical face and chamfer to make it easier to measure the opening 3y size while having a chamfer to allow for maximum range of motion of a bone screw or spinal implant. The head of the bone screw or spinal implant 1 fits within the spherical seat within body 3. As the head of the bone screw 1 is larger than opening 3y, it becomes clearer from this view that the rationale behind the slots is to allow enough flexibility for the insert 3 to be able to expand enough to accept the bone screw or spinal implant head in a preferably elastic or partially elastic mode. This insures that the insert 3 will expand to accept the larger bone screw 1 head and then return back at least most of the way to retain the bone screw 1 head.

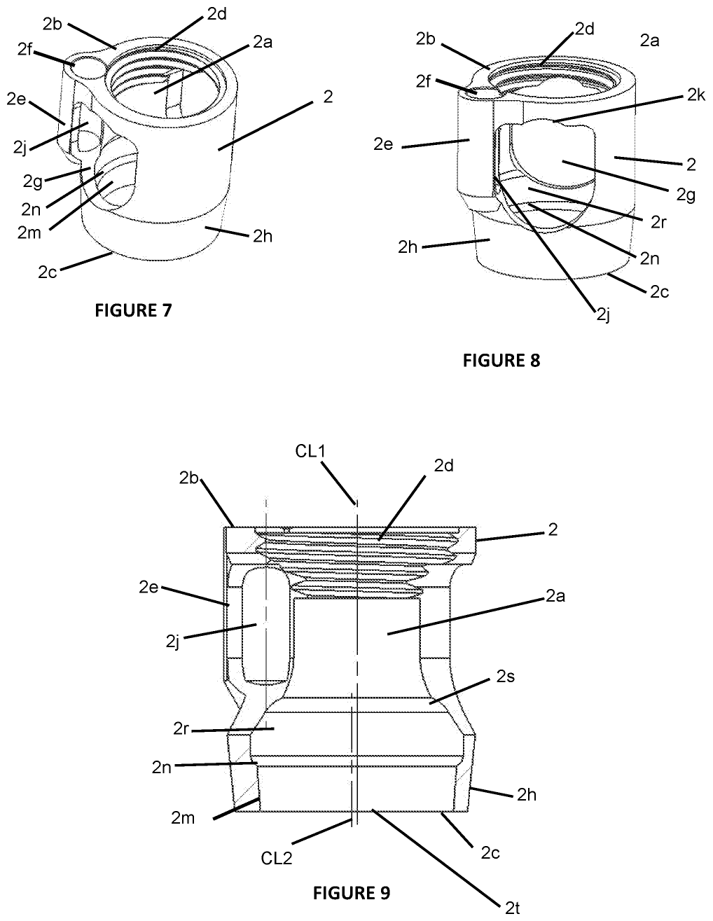

In FIGS. 7 and 8, the body of one embodiment is shown. This body 2, in addition to the features previously described above has a bore 2a within body 2. The top of bore 2a can be stepped or smaller in diameter than the center section to allow for the threads 2d to have a smaller minor diameter. This can reduce the outside dimensions of body 2 by retaining maximum body wall thickness. The bottom of the bore 2a opens into a recess 2r, a chamfer 2n, and a taper 2m. The instrument opening 2f intersects the inner body wall and cut through, leaving an opening 2j. This allows the instrument access to the gear rack, which will be detailed in drawings to follow.

FIG. 9 is a cutaway view of the body 2 to help clarify the details of its structure. As can be seen, the recess 2r is larger than the top diameter of the taper 2m, which allows room for the insert to expand when accepting the head of a bone screw or spinal implant. It is preferred to avoid any sharp edges, especially in titanium which is notch sensitive, so blend radii 2n between the taper and the recess and 2s between the recess and the bore 2a are provided. The taper intersects at the lower surface 2c to create an opening 2t. Also, it is possible to offset the bore 2a, thread 2d, taper 2m, and related internal features on a different center line than the exterior of body 2. By offsetting the center line C12 of the internal features relative to the external body C11, the material thickness of body can be directionally altered. This option allows an embodiment to shift more material to the side of the instrument opening 2j. In addition, this option, or absence thereof, allows to control the wall thickness of the taper feature. By increasing or decreasing the chamfer or taper 2h relative to the internal taper 2m, the wall thickness can be increased or decreased. Offsetting the internal features relative the external body wall, allows for thinning one wall section relative to another. The reasons for controlling wall thickness will be best understood during the discussion on this embodiment's assembly and locking.

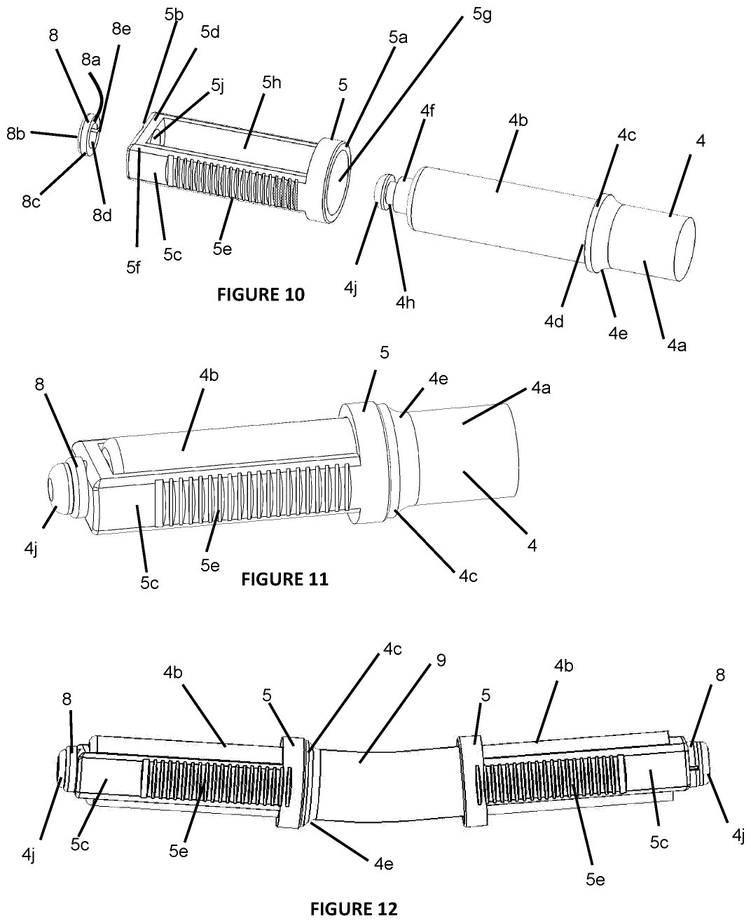

Shown in FIG. 10 is an embodiment of a rod assembly as briefly discussed earlier. The rod assembly consists of a rod 4, a rack section 5, and a snap ring 8. The rod 4 has a first section 4a for connection with other implants or polyaxial screws, a cylindrical rod portion 4b, a collar 4c larger in diameter than cylindrical rod portion 4b, creating a lip 4d. A blend radius 4e prevents the risk of a stress riser to the rod member at the collar 4c location. The round rod portion 4b has a reduced diameter extension 4f, a groove 4h, and an end 4j, that can be rounded to avoid soft tissue impingement. The rack section 5 consists of a first face 5a and an opposite second face 5b, having an external surface 5c having part of the surface cut away to create flats 5d, the external surface 5c having features such as teeth to create the equivalent of a gear rack over at least part of the length of the external surface 5c, and blend radii 5f around the edges. The rack section 5 also has an internal bore 5g extending through face 5a, which, which cut by flats 5d, create two internal rounded surfaces 5h in the locations where the flats occur. A hole through the second face 5b extends through to the inner bore 5g. A snap ring 8 is also provided having a face 8a, an opposing face 8b, and exterior surface 8c, an internal bore 8d, and a slot 8e to allow the snap ring flexibility to open and close.

Now with the individual components detailed, the rod assembly shown in FIG. 11 can be understood more clearly. The rack 5 is slid over the cylindrical rod portion 4a until fully seated with the face 5a against face 4b. This will allow the rod section 4f to extend through the rack face 5b, exposing the groove 4h. This will then allow the snap ring 8 to be slipped into groove 4h, thereby securing the assembly in place. After assembly, the rack 5 can freely turn around the rod cylindrical section 4b. There are other ways to secure the two components so they remain assembled, including peening, welding, press fit sleeve or collar, threads, c-clip, or other mechanical locking mechanisms. It should be noted that the end of the rod assembly is shown here as cylindrical section 4a can be of any length, straight, contoured, or end in other geometries, such as spherical, oblong, square, or any other shape. The rod component 4 as a whole or in part can be contoured as well, not just section 4a. The rack 5 is located on the rod in different locations to provide compression and distraction capability where needed.

In FIG. 12, one variation of a rod assembly is shown as rod assembly 9, whereby both ends have one complete set of rod assembly features. Thus, there are two racks 5, one on each end that can freely rotate about a cylindrical section 4b. The rod section in the middle can be curved or straight. In this example, the center rod section is curved. Note also that one rod assembly end has a collar 4c and snap ring, and the other side only has the snap ring. This is to show that variations in the design are possible and within the scope of this invention. The snap ring, or equivalent retaining mechanism alone will retain the rack member 5 on the rod 4.

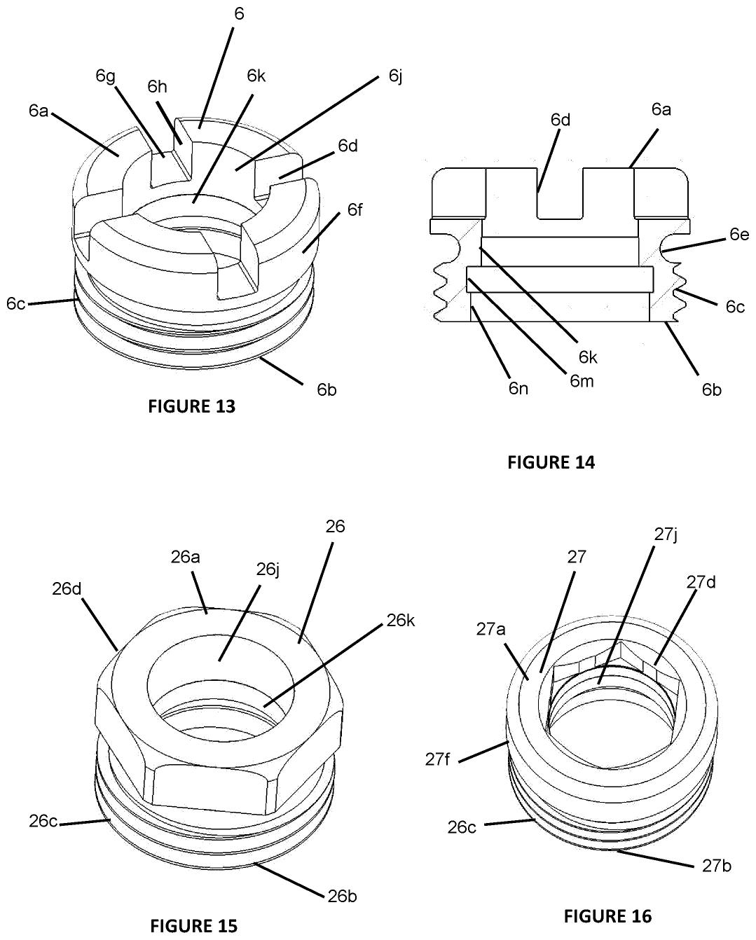

FIGS. 13 and 14 show the details of one locking nut embodiment. The locking nut, as described briefly before, includes driving features 6d, which in this example are slots having a driving face 6h and a bottom face 6g. The internal opening has an upper bore 6k, a recess 6m to accept a snap ring, and a lower bore 6n extending upwards from lower face 6b. The outside 6f in this example is round, but can be other shapes. While the internal features are designed to fit the insert 3 and snap ring 7, these features can be different, such as a single diameter bore with or without the snap ring.

FIGS. 15 and 16 show alternative locking nuts. In FIG. 15, the driving features of locking nut 6 are replaced by an external surface feature. In this example, the locking nut 26 has an upper surface 26a, lower surface 26b, threads 26c, and an outside surface with flats 26d. The bore 26j can have a groove 26k for accepting a snap ring when needed. In FIG. 16, locking nut 27 has an upper surface 27a, lower surface 27b, threads 27c, and an internal hexagon 27d for connection with an instrument. The bore 27j can have a groove for accepting a snap ring when needed. Of course, rather than a hexagon internal or external feature, these features can be any surface shape that can engage with an instrument in such a way as to allow the instrument to turn the nut. This includes additional flats, torx, etc.

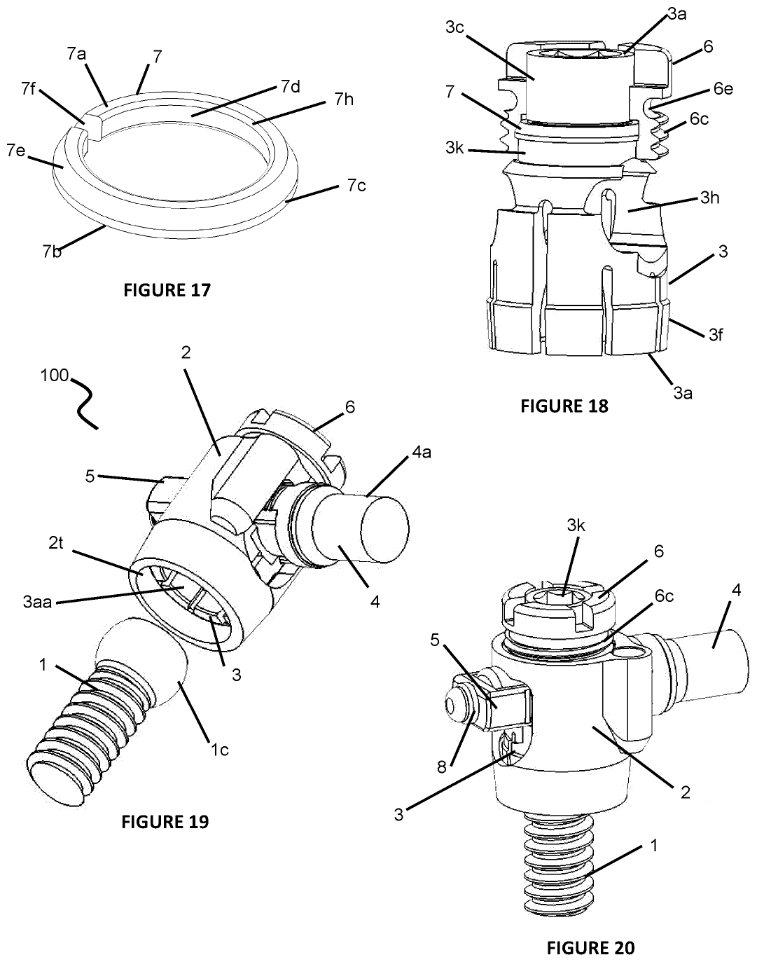

FIG. 17 shows an embodiment of snap ring 7. The snap ring, or also called a retaining ring, has an upper surface 7a, a lower surface 7b, an external diameter 7c, and an internal bore 7d. A chamfer 7e extends from the top surface 7a to the external diameter 7c. A slot 7f allows the ring to be able to flex open and be compressed inward. There are other forms of the snap or retaining ring that will work, such as removing the preferred chamfer 7e.

With the insert 3, locking nut 6, and retaining snap ring 7 now detailed, FIG. 18 shows how these components fit together. The retaining ring 7 fits within the insert 3 groove. When the locking nut 6 is pressed downward over the top surface 3a of the insert 3, the insert 3 will align with the retaining ring 7 held within the groove and the retaining ring 7 will snap into the groove in the locking nut 6. This creates a sub-assembly whereby the locking nut 6 can freely turn around the insert top section 3c, but is axially constrained.

As shown earlier in FIG. 3, the components in the exploded view in this embodiment are correctly shown in the direction of assembly. The retaining ring 7 is placed into groove 3j on insert 3, and the insert and retaining ring assembly is inserted into and through the bottom of body 2. Insert 3, due to the slots 3g and 3x, is flexible enough to be compressed into and through the body taper opening 2t, which is smaller in diameter than the top of insert taper 3f. With the insert 3 now inside body 2, the locking nut 6 can be threaded until it is partially engaged with threads 2d in the body, and the insert pushed upwards until the retaining ring 7 engages the locking nut groove 6k. This effectively creates a single assembly whereby the three components are tied together. By allowing the locking nut 6 to turn yet be constrained in this manner, the body 2 can be forced to move up or down relative to insert 3 simply by turning the locking nut 6.

The complete assembly 100 shown in FIG. 19 shows the spinal implant or bone screw 1 with a spherical or semi-spherical head 1c, body 2, insert 3, rod 4, rack 5, and lock nut 6. The insert 3 is in the retracted position, which places the insert taper 3f in the larger bore 2r in the body. As discussed previously, the lock nut 6 is connected to insert 3 by the retaining ring 7. Thus, the position of the two is related. With insert 3 in the retracted position, the locking nut 6 is in its highest position relative to the top of the body, or most amount of threads exposed.

FIG. 20 shows the assembly 100, as in FIG. 19 with the insert 4 in the retracted position with the head 1c of the bone screw engaged within the insert seat 3aa. As the insert is in the retracted position, the insert rod opening is in the highest position. Therefore, the rod assembly 4 and rack 5 are also in the highest position relative to body 2. In this position, the body can be angled in any desired position relative to the axis of screw 1, as the assembly will pivot around the spherical screw head.

By tightening the locking nut 6, the body is drawn upwards. This motion compresses the taper 2m against insert taper 3f, which then compresses the insert spherical seat 3aa against the bone screw head 1c. At the same time, the lower surface of the locking nut 6b contacts at least a portion of rod 4, in the cylindrical section 4a, compressing the rod against seat 3p in the insert. This simultaneously locks the rod assembly and bone screw in the preferred location. Note that the locking nut 6 is compressing the rod 4, which is preferred, not rack 5. By locking to the rod section, a normal spinal rod that fits within the seat in insert 3 can also be substituted without effecting locking. The amount of force or torque it takes to turn the locking nut to lock the entire assembly 100 can be determined and optimized from design and testing. Partial locking can also be accomplished by turning the locking nut 6 at some force or torque less than that needed to fully lock the entire assembly. Thus, body 2 can be partially locked at a desired angle relative to bone screw 1 prior to fully locking the position of the rod assembly within body 2 and insert 3.

During locking, it is necessary to provide torque to turn the locking nut and lock the assembly and counter torque to balance the torque. Without the counter torque, the assembly will want to turn in the direction of the torque, exerting a significant force that would be deleterious in the spine. Almost all prior art systems use an instrument that goes over the outside of the polyaxial screw, thereby increasing the diameter of the implant and the amount of clearance a surgeon needs to allow to implant it. However, the novel implant described here uses a different approach. As described above, the insert has a feature 3k for engagement with a counter torque shaft. This eliminates the need for an external instrument, and allows for a small instrument to provide sufficient counter torque. For minimally invasive applications, where incision size is important, this feature provides significant advantages.

The additional benefit of the retaining ring 7 is now easier to explain and important when the need for loosening or revising the implant is needed. Without the retaining ring 7, which is a possible embodiment, loosening locking nut 6 will remove the locking nut 6. It will not disengage taper 2m of the body from taper 3f in the insert. However, with the retaining ring, loosening the locking nut 6 will force the body 2 downwards to disengage the tapers. The threads provide significant mechanical advantage to help drive the tapers apart. Once the tapers are free and the insert is fully retracted, the body and insert assembly can be removed from the screw head or spinal implant or repositioned and relocked. As revision and removal of implants within the body are important considerations, this technique and design allows for a much easier procedure. In addition, once the locking nut is loosened, the rod assembly or rod is also free to be repositioned or removed.

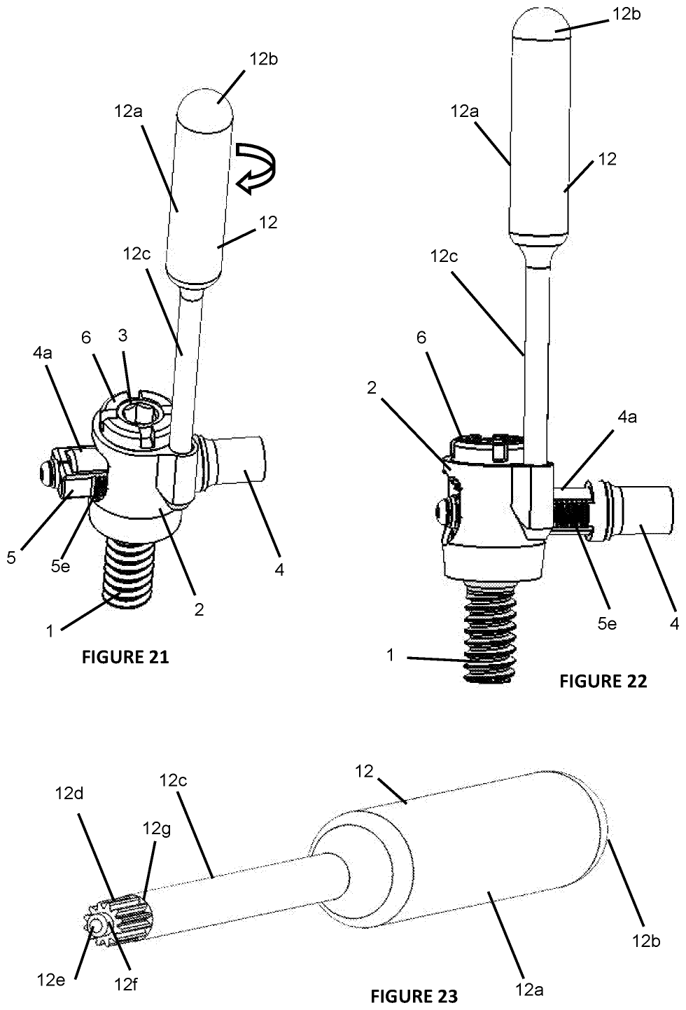

In FIGS. 21 and 22, one of the main benefits of this design is shown along with the instrument 12. The purpose of the rack 5 and the hole 2f in body 2 is to allow an instrument to enter hole 2f and be able to actuate rack 5. By turning instrument 12 in one direction the rack is moved in the same direction. In FIG. 21, the rack 5 is to the left. By turning instrument 12, the rack can be moved to the right, as shown in FIG. 22. Instrument 12 has a handle 12a with end 12b, and a shaft 12c.

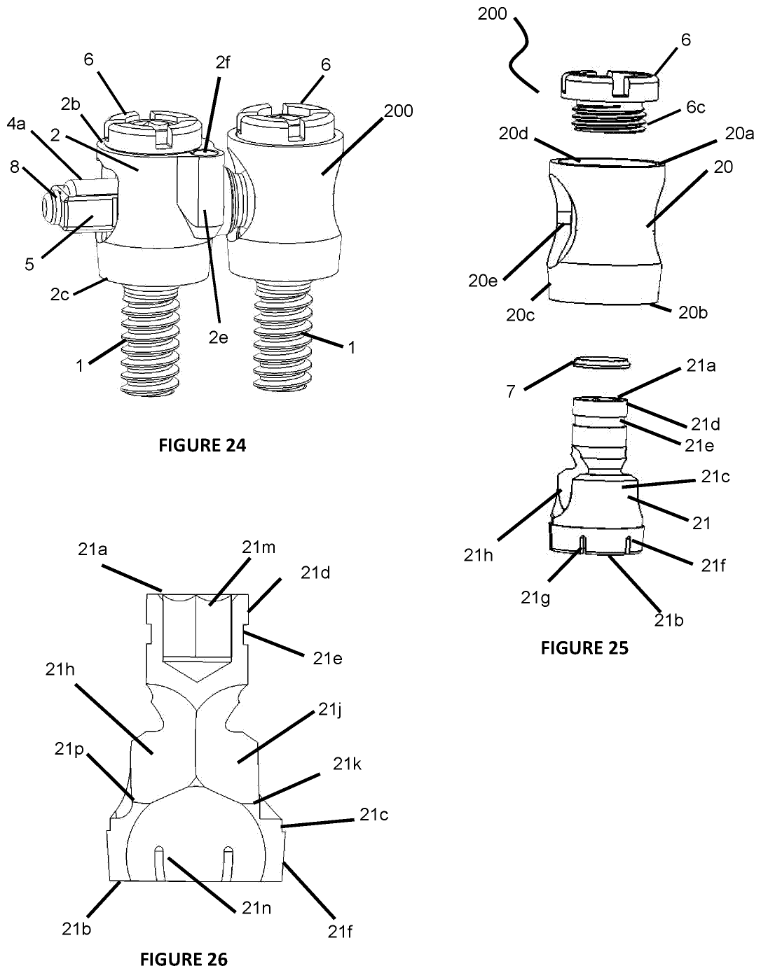

In FIG. 23, the details of the tip of the instrument are shown. The tip has gear teeth 12d that match the pitch and number of teeth to the rack, such that by turning instrument 12, the teeth engage rack teeth 5e to move the rack in at least one direction. It is, of course, preferred that the instrument be able to move the rack 5 in both directions. Instrument 12 has gear teeth with a first starting end 12e which can be, if needed, a pilot point for alignment within the body hole 2f, and an overall depth 12g. When the teeth are cut, it is likely the teeth at 12g with end in a radii due to the machining process.

Where the benefit of this approach can be seen is when at least two implant assemblies are placed in the spine, such as in FIG. 24. While FIG. 24 also represents a different embodiment which will be explained in the following drawings, the compression and distraction approach is the same. If the polyaxial assembly 15 with bone screw 1 is attached to the spine and, for this example, fully locked so that the angle and location of assembly 15 is effectively ridged, then moving the rack 5 with instrument 12 will move body 2 and the insert 3, lock nut 6, retaining ring 7, and bone screw 1. Of course, since the bone screw would be in the spine as well, moving the rack moves the spine. This is the principle of compression and distraction, whereby the vertebral bodies can be distracted, or spread apart, or be compressed and brought closer together. Normally this is done with large bulky instrumentation. The gear and rack approach allows for a very small instrument to provide a much better result with less force, more accuracy, and smaller incision. The amount of compression and distraction is only limited to the length of the rack, which can be any desired length as needed or preferred. In addition, this construct allows for a very small implant which is far more capable then current implant systems.

In further discussion of FIG. 24, assembly 200 is another embodiment of the implant without a rack accepting feature, but having similar components. In single level cases where only two polyaxial screw assemblies are required per side of the spine, only one rack 5 is usually necessary. Of course, two rack assemblies and the same body can be used, such as in longer constructs such as a corpectomy where additional distraction might be required. Where only one rack is required, two identical assemblies can be used; however, the polyaxial screw assembly does not need the exact same features.

As shown in FIG. 25, embodiment 200 has a body 20 having a upper surface 20a, a lower surface 20b, a lower section that can be cylindrical or tapered 20c, a threaded upper section 20d, and a rod opening 20e. The locking nut 6 is approximately the same as the one used in embodiment 100. The insert 21 has an upper surface 21a, lower surface 21b, a cylindrical section 21c, an upper cylindrical section 21d in which a groove 21e is cut into it, a lower tapered section 21f, and slots 21g to allow the lower tapered portion to be flexible. As per the previous embodiment, the slows allow for the insert to expand to accept a bone screw head or spinal implant as well as be compressed around the head to lock the angulation of the body relative to the bone screw or spinal implant. The rod opening 21h in this embodiment is different and actually a rod seat, which will be better seen in FIG. 26.

FIG. 26 shows a section view of insert 21. Seats 21h and 21j are spherical or semi-spherical to accept rod ends that are also spherical or semi-spherical. The openings 21k and 21p of the spherical cuts 21j and 21h into insert 21 create openings that accept the spherical rod ends, but are smaller in diameter than the spherical diameter of the rod ends. This allows the rod ends to snap into insert seats 21h and 21j and be retained within. To accomplish sufficient flexibility, at least one of the slots 21g is long enough to extend from the bottom surface 21b until it intersects the spherical seats 21h and/or 21j. A feature 21m for engaging an instrument is provided for, and can be a hex, torx, square drive, or other engagement feature.

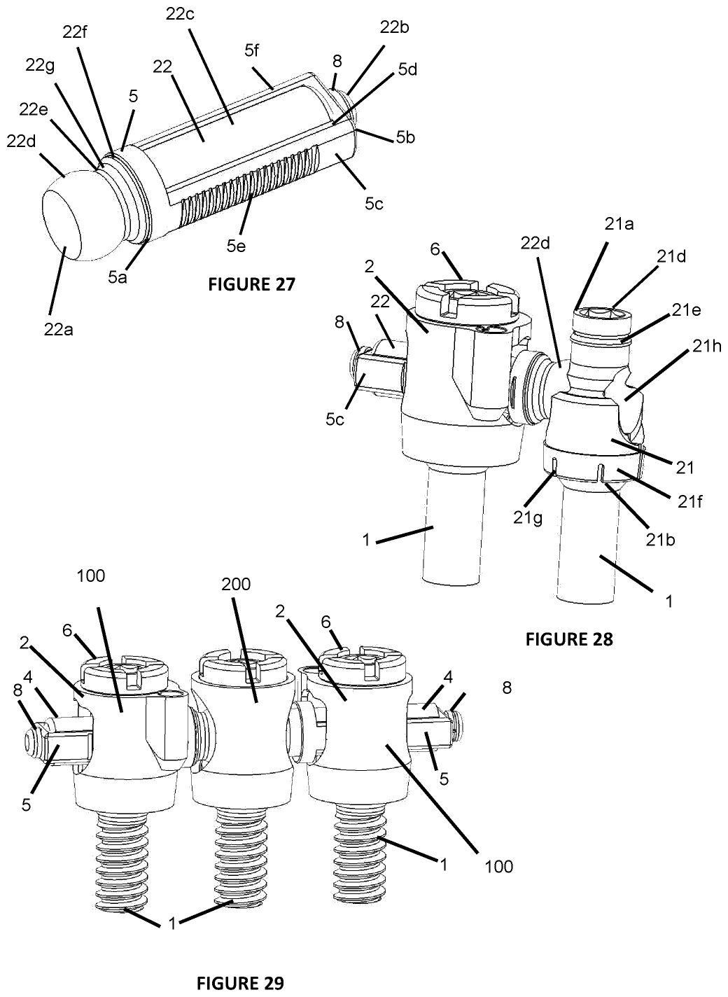

The rod assembly used with embodiment 200 shown in FIG. 27. This is approximately the same as the one shown in FIG. 10, with the exception that the end of the rod is replaced with a spherical end 22d. For clarification, new numbers have been assigned to the rod portion. The rod section 22 consists of a first end 22a and a second end 22b. A rod section 22c is connected to a collar 22f and blend radii 22e and 22g. There is a groove, in which the snap ring 8 fits to hold on the rack component 5, which has been previously described.

FIG. 28 shows the spherical end 22d snapped into insert 21. Note that in this example, the second spherical seat 21h is open. While in a single level construct to fuse two vertebrae together, the second seat is not required. However, it is preferred to leave a second seat for potential future revisions. This allows a surgeon to add on to the construct by inserting another spherical ended rod or rod assembly into the seat without having to remove components from the body.

FIG. 29 shows a two level construct, whereby two rod assemblies 4 with racks 5 are attached to a central body assembly 200. This central body assembly, as shown and described in detail (FIG. 25) accepts two spherical rod ends, allowing for an additional spinal level or vertebral body to be connected via a bone screw or spinal implant, to assist in fusion.

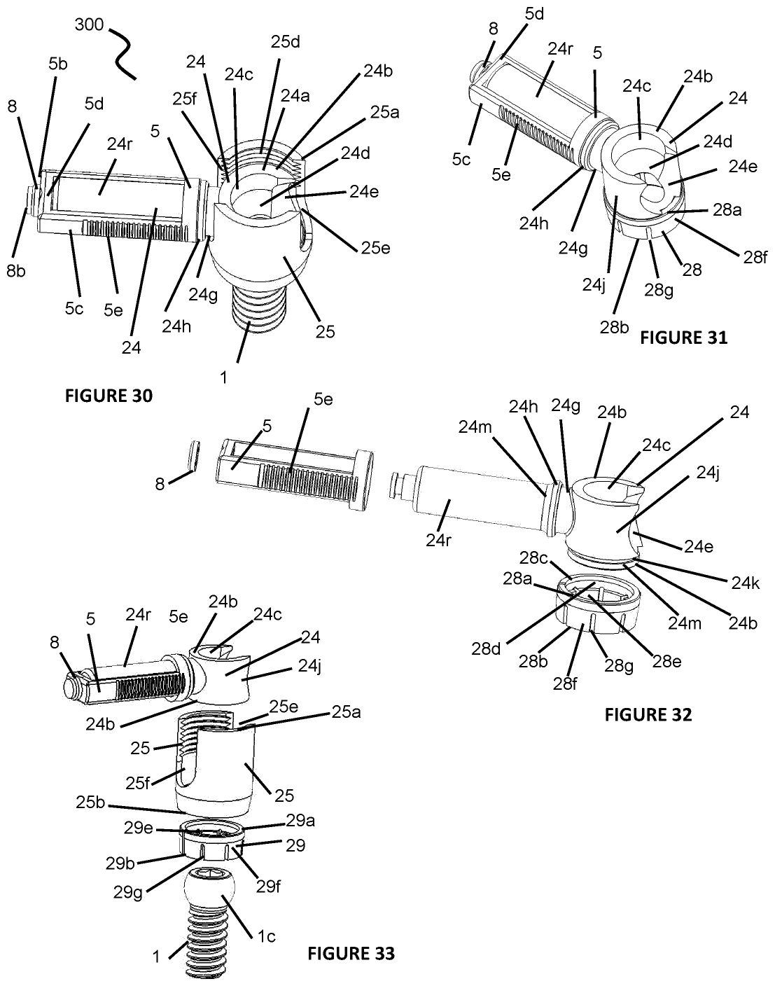

Shown in FIG. 30 is another embodiment 300 of an assembly. This embodiment is similar in that is uses a rod assembly having a rack section 5 as before with teeth 5e; however, the rod portion 24 has a different end. This rod 24 has an end 24b having a generally cylindrical portion with a top face 24a, a bore 24c, a spherical seat 24d, an opening 24e, and a generally cylindrical section 24r to fit within rack 5. A different body 25 is used for rod 24m having a top face 25a and an opening extending from the top face with threads 25d. The cylindrical end 24b has sufficient clearance with the threads 25d to enter from the top and be seated within body 25. A rod slot or opening 25f allows rod 24 to enter the body from the top, and a second seat 25e can be provided for adding an additional assembly at the time of surgery or in the future. Of course, this opening is not necessary for a single level 2 screw construct. The diameter of neck 24g is at least small enough to enter the body opening 25f. The larger the opening 25f compared to the neck diameter 24g, the more the rod portion 24 can move or turn relative to the axis of body 25. A collar 24h allows for the rack 5 to act as a stop as well as a smooth blend for the rod and rack assembly.

In FIG. 31, the cylindrical section 24j also has a feature at the bottom for retaining a collet, in this embodiment a groove, and forms an assembly with the collet 28 having at least one slot 28g to allow the collet to expand and contract to accept bone screw 1. The opening 24e can be seen here to be conical, to allow a second spherical interface to fit within seat 24d and allow a rod or neck portion to have enough clearance for polyaxial motion.

FIG. 32 provides more detail on the rod cylindrical portion 24 and collet portion 28. The cylindrical section 24j has a cylindrical section 24m in which is cut a groove 24k. The collet 28 has a top surface 28a, lower surface 28b, a bore 28c, and a groove 28d. A spherical or partially spherical seat 28e designed to accept the spherical head of a screw or spinal implant allows the head 1c to be inserted into collet 28. The exterior surface 28f is tapered. Slots 28g provide for flexibility of the collet. These slots can be of differing heights, and one slot can be a through slot to provide more flexibility. The cylindrical portion 28c fits into the groove in cylindrical portion 24k. This retains collet 28 but does not restrict its ability to expand. It is preferred, but not necessary, to include clearance at the interface of 28c and 24k, such that compression of the taper in body 25 against collet 28 will be able to compress the collet at the top and the bottom.

In the preferred embodiment, the body 25 can also retain the collet 29 within the body without being directly connected to the rod cylindrical portion 24f. This embodiment is shown in FIG. 33. This collet can be inserted from the bottom of the body or in another embodiment, inserted from the top.

As shown in FIG. 33, when inserted from the bottom, the configuration is similar to body 2, where a lower taper has at least a large enough opening to accept the collet when the collet is compressed. The collet 29 has an upper surface 29a, lower surface 29b, a tapered surface 29f, and a spherical seat 29e to accept a bone screw head. The collet 29 is inserted through an opening in the lower face 25b until it enters a section within body 25 where the insert has sufficient room to expand upon insertion of the bone screw head. When the rod 24, rack 5, and snap ring 8 assembly is inserted into the body, the lower surface 24b contacts the top 29a of collet 29. When a lock nut or set screw engages the threads and is tightened, the rod assembly is forced downward, which compresses the collet into the taper within body 25, thereby compressing the collet seat 29e against the screw head 1c, which locks the assembly and screw angle.

When inserted from the top, the collet can also be retained by entering a section or pocket that that the compressed collet can then be allowed to expand outward. An alternative way is to provide for a retaining ring within the body that can be pressed or snapped into position to hold the collet within the body.

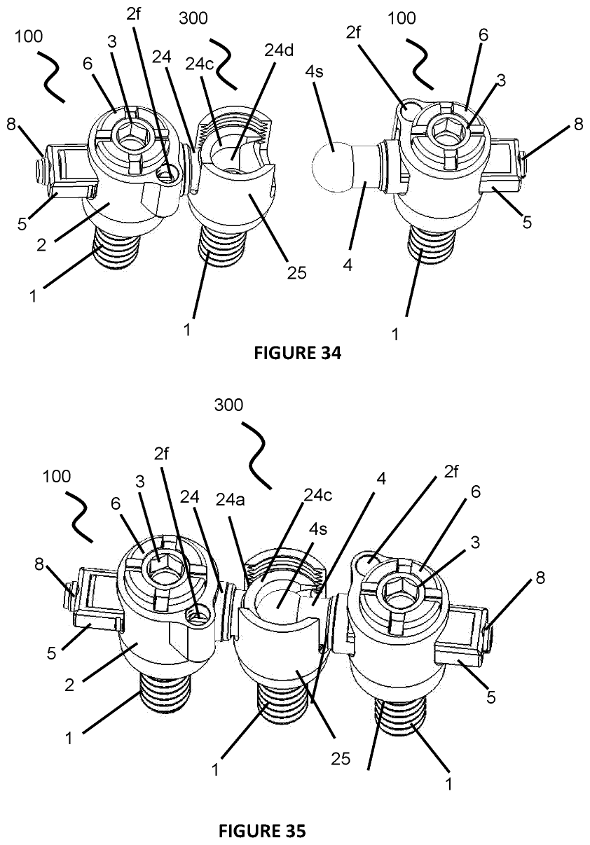

In FIGS. 34 and 35, the advantages of combining some of the embodiments can be better understood. The assembly described as 100 in FIGS. 1 through 23 is combined with the assembly shown in FIGS. 30-32. In the use of normal polyaxial screws, a single rod would connect all three screws. However, by using the described approach, the procedure is broken down into small segments that allow for easier placement of small rods. This is extremely helpful for minimally invasive procedures. More specifically for this embodiment, FIG. 35 shows how the spherical head 4s of a rod assembly (FIG. 2) can fit within the spherical seat in rod member 24. By using a lock nut or set screw, a single linear load created by advancing the locking nut or set screw by the threads presses the rod head 4s against seat 24d, which in turn presses the tapered collet 28, or 29, depending on the embodiment against the screw head as the taper is compressed against the taper inside body 25. This creates an efficient way to lock the assembly. Basically, the ball and socket arrangement creates a single rod that can be locked at the connection point. Note that if the head 4s is not present, the assembly would still lock. The locking nut or set screw would press against the top 24b of rod 24, creating the downward force to lock the assembly. This allows for a single level procedure to be done, leaving the seat open for future additions. This seat can be left open or blocked or closed off to prevent bone and soft tissue from blocking the seat over time. Furthermore, with this double rod/rack construct, the two racks obviously double the amount of compression and distraction that can be achieved over a single rack construct.

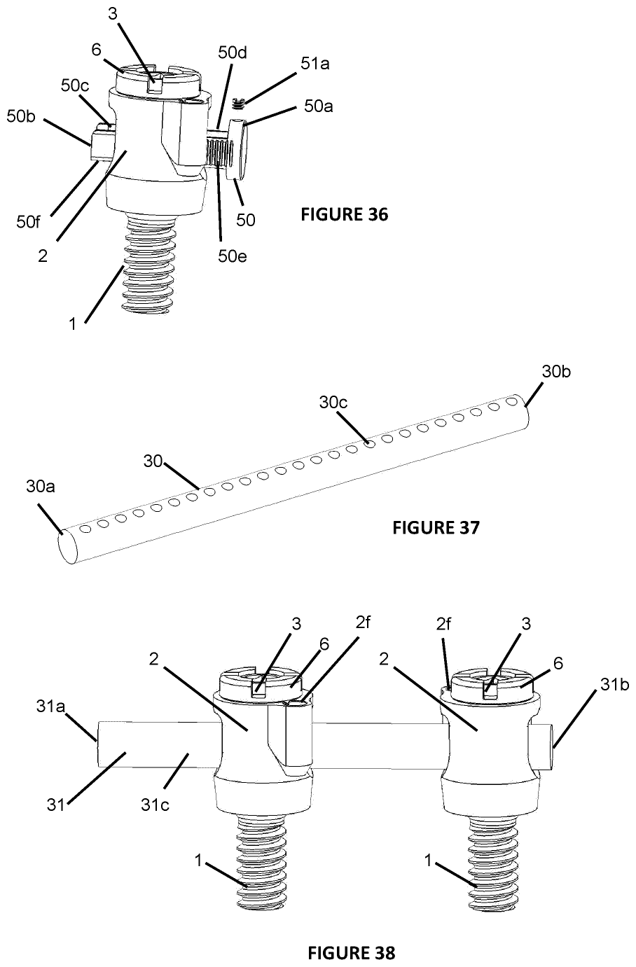

In FIG. 36, rack component 50 is similar to the previous rack 5; however, this rack component can be positioned on a rod in any desired position and secured into place. As can be seen, the rack component 50 has a first end 50a, a second end 50b, an inner bore or opening to accept the rod, a top surface 50d, teeth 50e, and a lower surface 50f. This rack component engages the same body 2 and insert 3 discussed previously. A locking mechanism, such as a set screw 51a, allow the surgeon to lock the rack component 50 in the desired position prior to exerting compression or distraction. In long constructs, such as 4 or more levels, it may be desirable for the surgeon to use a single rod that he can use straight or contoured and allow multiple rack components 50 to be placed where needed.

In FIG. 37, a rod 30 having a first end 30a and second end 30b has detents 30c for the rack 50 to engage with to help lock the rack 50 from slipping. Obviously, there can be more detents in more positions axially and radially around the rod. However, creating stress risers in a high stress spinal rod is not preferred. The same can be said for foregoing the rack component and placing teeth in the rod. While simpler, the notches would create stress risers most likely leading to premature rod failure. It is better to have a smooth rod or one with minimal stress risers. The rod shown here is simply a possible embodiment showing potential options.

FIG. 38 shows two assemblies as generally described as 100, but without a rack component. These assemblies are shown on a normal rod 31 having a first end 31a and a second end 31b. The geometry of normal spinal rods 31c is generally round. However, other shapes have been used, and the seat of the implant is capable of accepting other shapes. This figure helps show the universal nature of the novel implant shown herein. The implant can be used in multiple ways according the needs of the surgeon.

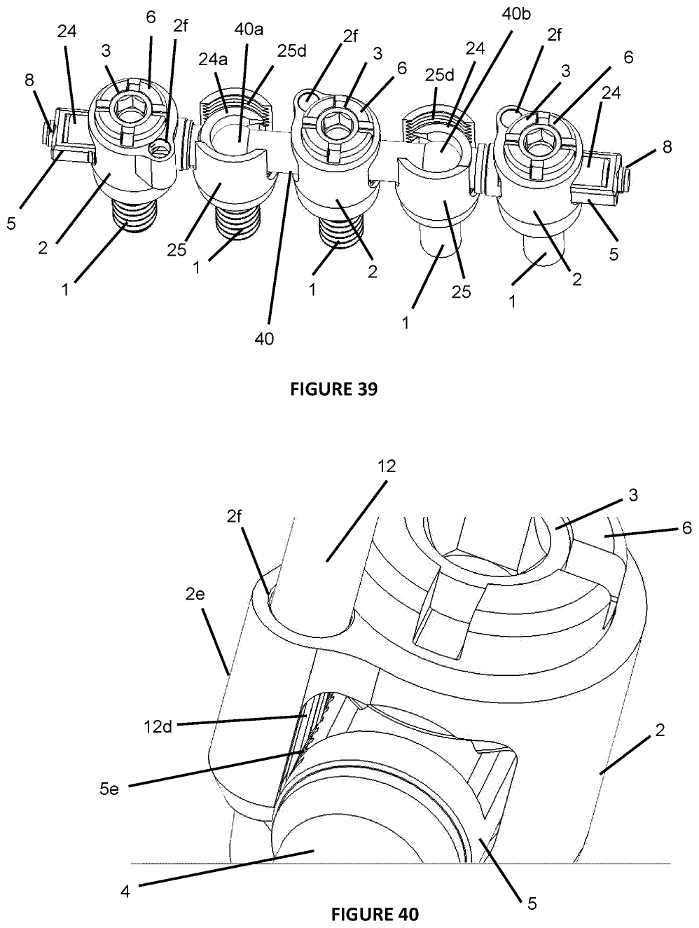

In FIG. 39, it is also possible to combine embodiments and connect them in different ways. In this example, a rod 40 has a first end 40a for engagement with seat 24a of rod assembly 24 inside body 25, and a second end 40b for engagement with a second body 25 and seat 24a. In this example, the ends 40a and 40b are spherical; however, they can be spherical, semi-spherical, or any shape that allows for at least one end of rod 40 to pass through insert 3. Obviously, there are other possibilities for connecting multiple components, such as having effectively two rod sections that connect together at one insert 3. One rod and can sit within the second rod end, such as in a telescoping arrangement, or two rods can sit side by side within the body assembly. In either example, the lock nut would exert sufficient downward force to lock the complete assembly.

In FIG. 40, to help clarify how the instrument 12 engages with the rack 5 and rod 4 assembly and similar embodiments, the instrument teeth 12d are shown in a close-up engaged with the rack teeth 5e. In practice, the instrument is turned until the rack is moved sufficiently in the desired direction to create compression or distraction, and then the lock nut 6 is tightened to hold the position. This is easily reversible by loosening the lock nut. Either the force from soft tissue will return the implants back to a neutral load position or the instrument can be used to readjust the location.

In surgical use, the most common application with this invention would be in the treatment of the lumbar or thoracic spine. Spinal screw assemblies or hooks are attached to a bone structure of the spine. This provides excellent visualization of the screw placement site and anatomy. As the implants described lock onto a spherical or semi-spherical head, a bone screw or spinal implant, such as a hook with a spherical head can be used. While the variations shown show multiple related options, it is easy to see how the features can be combined when multiple level polyaxial assemblies are used.

When spherical head bone screws are used, polyaxial assemblies are snapped over the bone screw heads. Of course, the modular nature of the design allows the polyaxial screw assembly to be attached to the bone screw head first, and the entire assembly implanted as one piece (the locking nut can be tightened to hold screw angulation during insertion); however, it is preferred to implant the bone screws or spinal implants first. In a single level case whereby only two vertebrae are to be fused, a single connector is used preferably on each side of the spine and right size selected. A polyaxial connector generally consists in this case to have two screw body assemblies and a single rack and rod assembly connecting the two. Either a measurement of the distance between the screws can be taken or a template used to determine the proper connector size. The rod and rack and its variations allow for a single connector assembly to cover a range of screw head distances, which significantly reduces inventory. Once the correct size connector is selected, the body assemblies allow the insert within to be snapped over the top of at least one of the screw heads, and the length adjusted until the other polyaxial assembly captures the second screw head. The lock nuts can then be tightened to lock the assembly or compression and distraction can be done. As the polyaxial screw assemblies, such as that shown in embodiment 100, adjust automatically to significant bone screw angulation, placement of the connectors is straightforward.

One of the key benefits of the rod and rack assembly approach shown is the ability to be able to use a small instrument to do full compression and distraction while minimizing the size of the polyaxial body. This allows the surgeon to work through a small opening or cannula and use the instrument to adjust the spine as needed. Turning the instrument provides linear motion, which is far more efficient than most of the instrumentation available today. Once the proper compression or distraction is achieved, the surgeon then uses an instrument to turn the locking nuts, which compress the lower surface of the locking nut against the rod, which then compresses the insert or collet taper against the taper in the body against the screw head, thereby locking the entire assembly.

It is important to note that the insert 3 has an interface 21m for engaging a counter torque instrument. During locking, the rotational force of turning the locking nut must be countered, otherwise it will want to turn the rod which in turn will transfer undo force against the spine. By exerting a force through the instrument to interface 21m, which can be a simple T handle on a shaft with a tip to engage 21m, the surgeon can offset the locking force and prevent loads from being transferred to the spine.

During any surgery or after the surgery, it is always possible that the surgeon may need to revise or remove the screws. By using a snap ring 7, the locking nut is fixed axially but can still turn. When the nut is engaged with the body, the body will then be moved up or down depending on the direction the locking nut is turned. Thus, if the assembly is locking by turning the locking nut clockwise, if the nut is turned counterclockwise, it will want to force the body downwards and away from the insert taper, thereby forcing the assembly to unlock. In many polyaxial screw designs, this is a challenging task and sometimes requires the surgeon to cut the spinal rod in vivo to remove the assembly.

One of the further advantages of the assembly generally shown in 100 is the ability of the insert and body to accept the rack and rod assembly, or a normal rod that is straight or contoured. This provides the surgeon significant options. He can use the implants to form a connector, or single level rod and rack construct, or place implants on a straight or contoured rod to cover as many levels are required, even correct scoliosis by well understood de rotation.

As shown, more than one rack can be used on a single rod construct, and a rack assembly can be placed independently on the rod, allowing additional options for compression and distraction in the case of long rod multiple screw constructs.

When using the assemblies generally shown in 200 and 300, it is possible to use a connector to connect two screws together while allowing for a connector to attach at least one more screw on a rod or rod and rack assembly, such as shown in FIG. 34. At the time of surgery, the patient may need only a single level fusion. However, the open seat exists to make adding an additional level fairly easy. Of course, this allows for a two level case be done as well as the primary surgery.

By using rods having ends that interface with the seats in rod 24, as shown in FIG. 39, two single level constructs, which have two bone screws for insertion into two pedicles, can be connected by a middle rod. Such a construct allows for the ends of the construct to have compression and distraction, and the middle rod to accept another body, rack, or simply span a distance, such as in a corpectomy procedure.

Thus, the embodiments shown here allow for a multitude of approaches. The bone screws and inserts may have small through holes for accepting a k-wire. This allows the assembly to be inserted over the k-wire once the wire is placed in the pedicle.

One of the additional benefits is that the locking mechanism is built into the assembly 100 and 200, which eliminate the steps and instrumentation required to place set screws. This also eliminates the risk of cross threading. Also, in example embodiments body 2 and 20, the body has an opening to allow the rod or rod assembly to enter, but does not completely split the side of the body, as in almost all polyaxial screws. This allows the body to be more rigid and prevents splaying of the body as the lock nut is tightened, increasing efficiency of the locking energy.

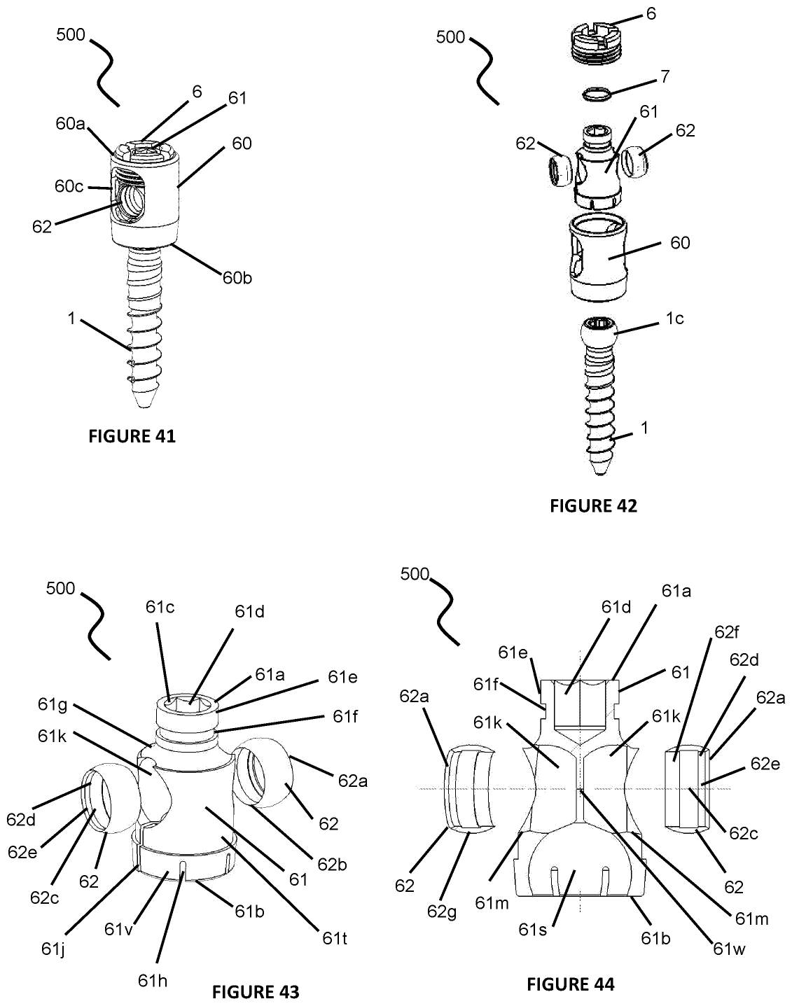

As shown in FIGS. 41 through 48, the basic concept of the polyaxial assembly described herein can be altered into another embodiment. The present invention provides generally for a spinal implant, including a body 60 having an insert 61 received within a bottom portion of the body 60, the insert 61 receiving a bone screw 1 at a bottom portion for attachment to a spine, a rod 65 received within at least one seat 61k on a side portion of the insert 61 wherein the seat 61k does not extend through a top surface of the body 60 or completely split a side of the body 60, the seat 61k including at least one bearing 62 that receives the rod 65, and a locking nut 6 received within a top portion of the body 60 and interlocking with a top portion of the insert 61 through a snap ring 7.

The general assembly, as shown as 500 in FIGS. 41 and 42, consists of a body 60 having a top surface 60a and a bottom surface 60b, an insert 61, at least one spherical bearing 62, a bone screw 1 with head 1c, and a lock nut 6.

FIG. 43 shows more detail of a dual spherical bearing version of insert 61. This insert consists of a top surface 61a, bottom surface 61b, an opening and chamfer 61c leading to a feature for engaging an instrument 61d. The feature 61d, shown here as a hexagon for engaging an Allen key, can be a torx feature or a number of other features for engaging an instrument. The upper section is a cylinder 61e with a groove 61f. A blend radius 61g prevents a stress riser and reinforces the section between cylinder 61e and the larger insert cylindrical section 61t. A taper 61v is machined into the insert. In this example, the taper is larger in diameter at the top than cylindrical section 62t for easier fit within the features of body 60. Grooves 61h and 61j are cut into the insert to allow the insert flexibility to accept the bone screw head as well as the flexibility to better compress against the bone screw head. These slots can be of varying height and width, and as shown in this figure, 61h extends upwards into spherical seat 61k. Slots 61h can also be longer than shown. The seat 61k is of a shape sufficient to retain bearing 62. This seat can be spherical and/or partially tapered to retain the bearing. Partially tapering the lower portion of the seat may provide better locking by increasing the interference of the bearing within the seat as the assembly is locked. The bearing 62 consists of a spherical or partially spherical external surface, a first end 62a, a second end 62b, and internal bore 62c a lip 62d and a second cylindrical section 62f. In this example, the bearing sections 62e and 62f are the same diameter and 62C represents a groove that is larger in diameter than 62e and 62f. The bearing would have a slot such that it can expand and contract to accept a rod having a cylindrical section that fits within groove 62c to help lock the rod within the bearing when the bearing is compressed by the lock nut during locking. Of course, this feature can possibly be omitted such that the internal bore of the bearing can be cylindrical and the rod one diameter. Also, rather than the bearing having a groove, the features can be reversed such that the rod has a groove and the bearing a positive feature or lip to engage the groove in the rod. Any bearings 62 can be machined or formed from a softer material than the locking nut 6.

FIG. 44 is a section view of insert 61 and shows more detail of a two bearing insert and bearing features. Spherical or partial spherical seat 61s fits over bone screw head 1c. A chamfer 61m at the opening of the bearing seat 61k allows the bearing and rod to have maximum angulation without hitting the side of the insert. The center wall 61w prevents the ends of two rods, one in each bearing from extending too deeply and hitting each other. Of course, with a feature in the bearing and/or on the rod, such as larger section on the rod, the location of the rod in the bearing can be controlled and the center wall 61w can be omitted. Furthermore, the center wall 61w can have a groove, either through or partially into the center wall, such that the end of the rods, when forced into the groove, remain aligned to each other in at least one plane. A vertical groove would aid in aligning the rods in one plane while allowing for lordotic correction. This can be ideal when trying to align the two rods in the sagittal plane for scoliosis correction. The spherical bearings 62 can be aligned side by side such that rods 4 inserted into the bearings 62 can overlap and by approximately parallel to each other.

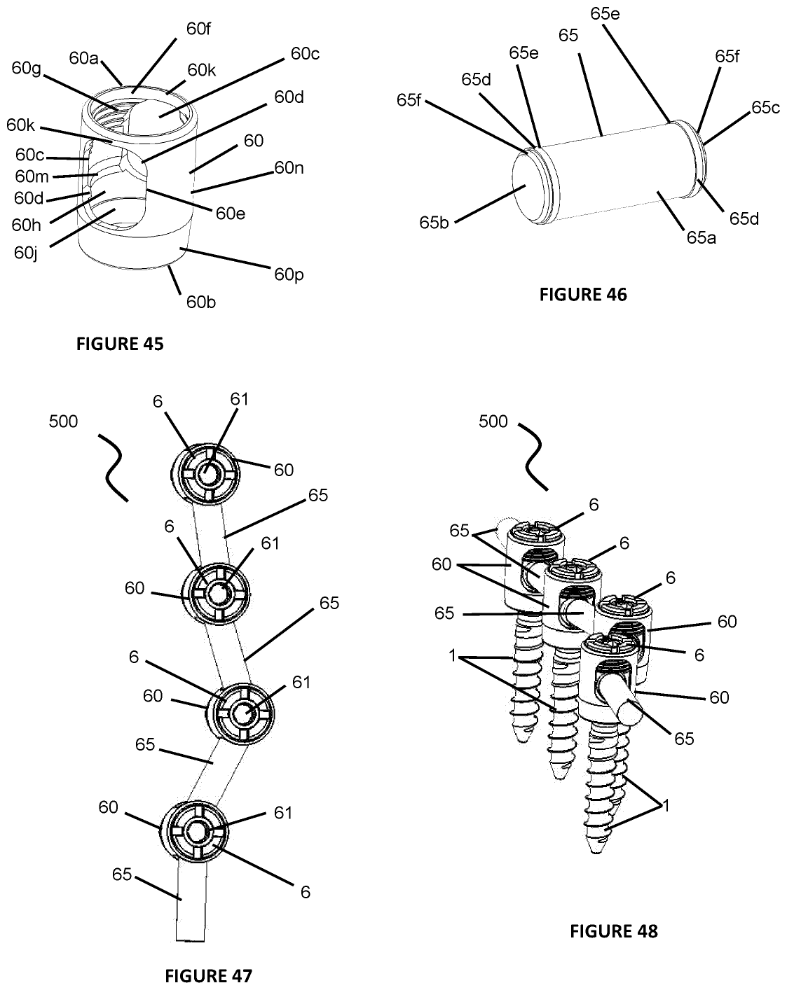

FIG. 45 shows the body 60. This body has a top surface 60a, bottom surface 60b, openings 60c for the rods to pass through, which have blend radii 60d to avoid stress risers and any sharp edges from contacting the rod. Note that the rod opening in this embodiment does not completely split the body at the top and leaves material across the top 60k. When polyaxial screw bodies have a complete split or opening for top loading of a rod, tightening a set screw splays the body open. The embodiment shown here completely avoids splaying and creates a more efficient approach to locking, maximizing the amount of energy that can be used to lock the assembly. Top opening 60f is larger than the lock nut to allow the lock nut to seat deeper within to reduce the overall profile of the assembly. A portion of bore 60h of the body has thread 60g to accept the lock nut. A lip 60m extends from bore 60h. While this lip is not necessary for overall function, it aids in containing insert 60 within body 61, as the lip inner diameter is smaller than the outer top surface of taper 61v. Thus, the insert 61 can be retained within the body and the body will not fall off if the threads of the lock nut and body are not engaged. The lower portion of bore 60h has a taper 60j for engagement with insert taper 61v. The outside of body 60 has a wall 60n. It is preferred to taper or chamfer the lower section of body wall 60n to allow for better fit within the spinal anatomy as well as to control wall thickness and flexibility of the body where the body taper 60j engages with insert taper 61v. Controlling wall thickness and allowing some flexibility under load can be important in compensating for the tolerances of machined parts, since parts vary in dimensions over a production run. While the body is shown in a generally cylindrical form, obviously it can be other shapes, such as rectangular, oval, or any other shape that allows for suitable performance.

FIG. 46 shows a small rod having features for engaging bearings 62. In this embodiment, a ring or larger cylinder 65d extends from the smaller rod diameter 65a. This ring can be close to both ends 65b and 65c. However, if the rod is used to engage another embodiment or other polyaxial screw, one side may have the features needed to engage the bearing and the other side simply a cylindrical portion. The ends of the rod are preferably chamfered 65f to allow for easier insertion with the bearing. The larger cylinder 65d creates a lip or edge 65e, which helps engage and lock the rod within the bearing. Rather than a cylinder that extends from the surface, the rod 65a can also have grooves that extend into the surface of the rod. With a bearing having reverse features and a lip that extends into the bearing, the lip can engage the groove, creating a similar way of retaining the rod within the bearing. Also possible is the use of a rod with no features if the locking force is sufficient to compress the bearing against the rod, or a rod with tapered sections at the bearing engagement end to assist in locking the bearing to the rod. While shown as a straight rod section, in practice, this rod would often be curved.

In FIGS. 47 and 48, the generally assembly 500 is shown in a multiple polyaxial screw assembly connected by rods. Note that in FIG. 47, the top assembly has only one rod, leaving one bearing open for later use, and that the lower assembly has a rod with a smooth section for attachment to another polyaxial screw with or without a bearing. Of course, this rod can be the compression and distraction rod of the other embodiment with the appropriate features on one end to engage the bearing. As can be seen in these two figures, the bearings allow for significant variation in angle between rods. The polyaxial assembly can pivot around the bone screw head and the bearings can accept rods at varying angles, and the pivot can be in all directions and planes (and generally can provide greater angulation than regular polyaxial implants). In a scoliosis case, the rods would be straightened relative to each other, thereby straightening the spine and the assemblies locked to hold the realignment. This can be done by use of an instrument that can engage both rods and move one or both independently to align the rods in the desired position. Of course, as the spine is straightened, the distance between the screws can change. Therefore, the bearing can allow for the rod to slide within to compensate for this dimensional change. This is done by allowing room between the back of the bearing and the wall 61w in the body or other bearing seat and providing proper features in the bearing and/or rod.

With FIGS. 41 through 48 and the features described within, it can be seen that the polyaxial assembly embodiment allows for attachment of multiple polyaxial screw assemblies of one or more embodiments using relatively small rods. By using the two bearing variation, it is easy to see how a surgeon could create an effective construct with as many polyaxial assemblies as needed, each connected by small rods. Also, a single level fusion can be done using the compressor distractor rod a polyaxial screw embodiment discussed previously to create a fusion while leaving one bearing open to add a second level either at the time of surgery or later if needed for a later revision. This second bearing can also be plugged to keep soft tissue and debris out until needed later. It is also possible to directly fix one end of the general assembly 500 to a rod or rod section directly to a second polyaxial screw assembly to provide a two polyaxial screw assembly to act as a prebuilt connector to connect two bone screws. In addition, it is possible to have only one bearing in assembly generally shown as 500 and the other side directly connected to a rod. This would also provide a connector to connect two bone screws while leaving the bearing side open to add another level.

For percutaneous spinal surgery where the bone screws and polyaxial assemblies are placed through tubes or very small incisions, connecting the assemblies with long rods is often difficult and restricts the number of spine levels that can be effectively treated. By using small rods, the approach is much easier. In addition, a level can be treated and additional levels added later. This has further advantages in complex cases, such as in the treatment of pediatric scoliosis.

In pediatric scoliosis, severe curvature of the spine can lead to significant health issues and must be treated. The current methods and implants for percutaneous treatment are very limited and usually require extremely long incisions due to the need for a long rod. By breaking the long into short sections and allowing sections to be added on as needed, a significant advantage can be provided. In addition, rather than having to preplan a long rod for correcting potentially more levels than needed, the embodiment shown herein with bearings can break down the long rod into small rods that can be done implant by implant. Thus, the surgeon can approach correction differently and treat the spine in smaller sections, adding on only as needed. It is understood that often correcting the apex, or most extreme part of the abnormal curve of the scoliotic spine can correct a significant amount of the deformity. By using the design disclosed within, the surgeon can treat only the apex to start and then assess the actual correction. If needed, more components can be added to extend the construct to complete sufficient spinal curvature correction. This is variation of the well understood translation technique for scoliosis curvature correction.

The assembly generally shown as 500 locks the rod or rods and bone screw by tightening the lock nut. The locking nut draws the body upwards to force the taper of the body to engage the taper of the insert while compressing downwards on the bearing(s). This creates an efficient loading model where the tapers compress the bone screw head to lock it at the proper angle while compressing at least one bearing against the rod. By using a polyaxial body having side openings that do not extend through the top of the body, the body is far more rigid. This prevents energy loss caused by splaying of open top loading rod slot body designs. While it may be possible to use the insert with a body with an open slot, it is not preferred, even if the design of the lock nut includes features to prevent splaying, such as an external collar that slides over a portion of the external wall of the body.

Note that the lock nut in general assembly 500 can also be a set screw and fully threaded with different engagement features to engage a driver. Also note that the insert feature 61d is designed to allow for exerting a counter torque force for the purposes of locking the assembly. In the case of a two bearing assembly with two unlocked rods and freely moving rods, it would be impossible to effectively lock the assembly by trying the hold the rods. Holding the insert steady provides excellent stability and counter torque when the locking nut is torqued to the proper locking load.

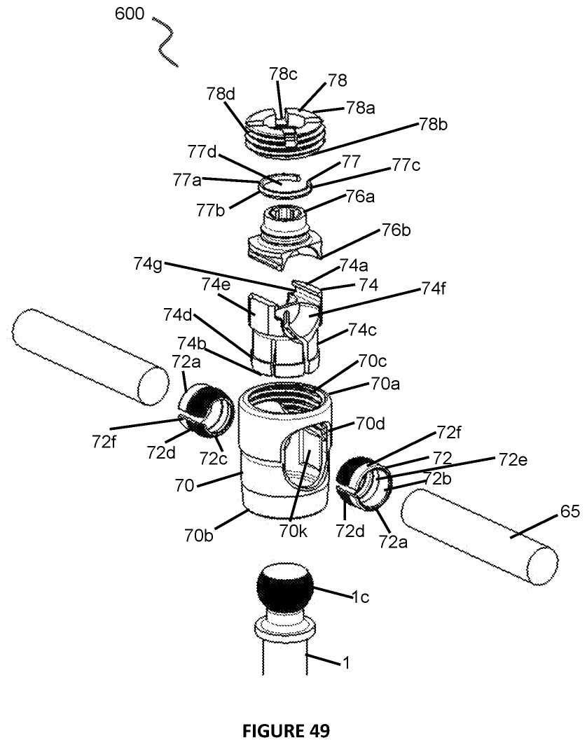

FIG. 49 shows a variation of general assembly 500, and is shown as general assembly 800. While similar, the insert is now in two pieces, with an upper section 76 and lower section 74. This allows for more uniform compression of bearings 72 during tightening of set screw 78. Body 70 has an upper surface 70a, lower surface 70b, a partially threaded internal bore 70c, and openings 70d for bearings 72 and/or rod 65 to extend through. The openings have clearance to allow the bearing to freely angle and rotate in one or more directions. The internal bore of the body also has a key 70k to maintain alignment with the flats 74e on insert 74. While the flats can be removed, doing so can allow the insert to rotate relative to the body, reducing angulation of one or both of bearings 72. The insert 74 has a top surface 74a, lower surface 74b, a center section 74c, and a tapered portion 74d. To allow the insert to be more flexible, 74c can be smaller in diameter than the top of the taper 74d. In addition, the larger top of the taper can be used as a feature to keep insert 74 within body 70 once assembled, as the taper top fits under keys 70k in the body. The taper top outer diameter in this case is larger than the width across keys 70k, such that the insert compresses and expands to fit under key(s) 70. While one key may be sufficient, two are preferred, one on either side 180 degrees apart. Insert top 76 has a top surface 76a and lower surface 76b. Two ribs or extensions 76n are designed to fit within groove 74g. There is clearance between groove 74g and ribs or extensions 76n such that insert upper section 76 can move up or down. Thus, when the bearings 72 are inserted into the assembled insert, upper section 76 is pushed upwards. However, when locknut 78 is tightened, it can exert pressure against upper section 76 to push it downwards to create compressive force against bearings 72, thus simultaneously locking the rods and bearing angulation in the desired locations. Upper section 76 also has a groove 76j for containing a snap ring. Snap ring 77 has an upper surface 77a, lower surface 76b, an external surface 76c that is preferably partially chamfered, and an internal bore 77d. This ring is split so it can expand and contract and fits within groove 76j of the insert upper section 76. The lock nut 78 has an upper surface 78a, lower surface 78b, a driving feature 78c, which in this example are slots but can be other features, external threads 78d. The locknut slides over top surface 76a of upper insert section 76 such that the snap ring slides within a groove in the lock nut. Thus, the lock nut is retained on the insert assembly. While this can be eliminated, and is not necessary to locking and function, this is important when trying to keep the locknut on the assembly as well as for disassembling the locked assembly. With locknut 78 attached to the insert, the locknut becomes integral and the surgeon does not have to add it or worry about it becoming loose. In addition, if it is desired to unlock the assembly, turning locknut 78 in the opposite direction of locking, the snap ring will allow the locknut to effectively pull the insert up to disengage the locked tapers of the lower insert section 74 and body 70. Revision of a system is always a concern and this provides a controlled way to allow this to occur. In the unlocked position, taper 74d of the lower insert section fits within a larger section of the body, thus allowing room for the insert to expand to accept head 1c of bone screw 1.

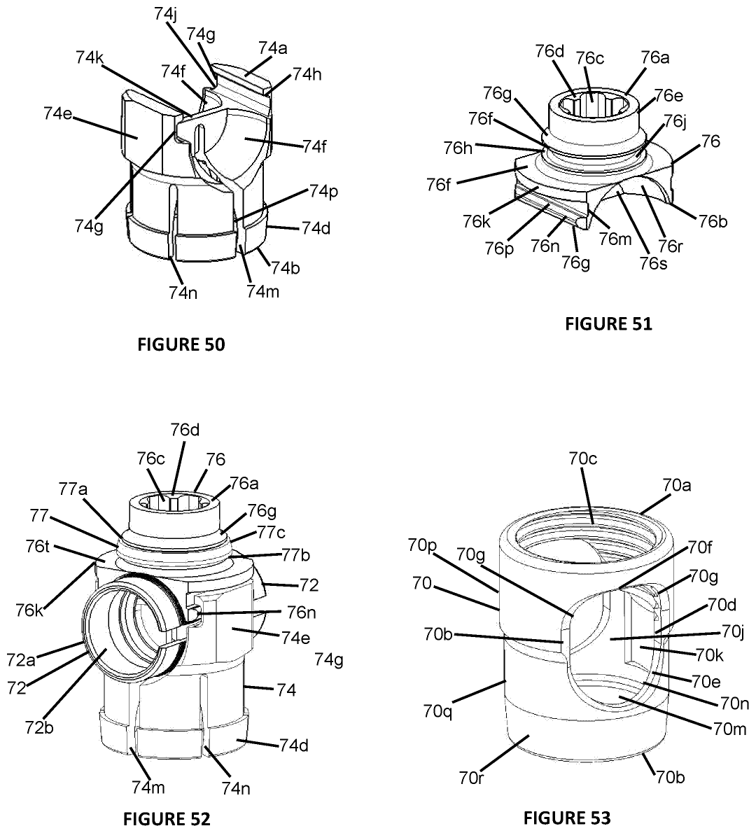

FIG. 50 shows the lower section 74 of the insert in more detail. As discussed above, section 74c, which is preferable cylindrical, is smaller than the top of taper 74d. This creates a lip 74e, which can hold the assembled insert in the unlocked position within body 70. Groove 74g also has radii 74h and 74j to reduce stress risers when the insert is pulled on during disassembly. At least one seat 74f for at least one bearing is machined or formed in the insert section 74. This seat can be partially spherical, partially spherical with a cylindrical extension 74r above the centerline of the spherical seat, or a different shape, such as an oval, tapered pocket, or other shape to retain and aid in locking the bearing(s). In this embodiment, there is a partially spherical seat 74f that is machined to leave a central wall 74s between two spherical seats. This central wall 74s allows a web to remain in the center of the part such that when a slot 74m is machined into the part, a long hinge can be formed to allow lower insert section 74 to be flexible enough to allow screw head 1c to fit within the seat by expanding the insert. The diameter of screw head 1c is larger than the opening of spherical seat 74p designed to accept screw head 1c. This allows for retention of the screw head 1c within insert seat 74p, rotation of the screw head within the seat, and excellent resistance to dissociation of the assembly under normal physiological loading. When the top of the lower insert section 74 is cut to create slots 74g as well as room for fitting and movement up and down of upper section 76, an edge 74k is created, which then forms the upper edge of the central wall 74s. By reducing the distance between the top of slot 74m and 74k, the stiffness can be altered to allow the bone screw head 1c to fit within without excessive force. Of course, it is preferred to create a balance as to avoid creating such a thin hinge as to create significant plastic deformation of the hinge area when the screw is inserted. Adding addition slots, such as 74n, allow the lower insert section to be flexible such that taper 74d can be compressed inwards against screw head 1c to lock the head to the insert at the desired angle.

FIG. 51 shows the upper section of the insert in more detail. The upper section has a cylindrical section 76e extending from top face 76a, which transitions into a larger cylindrical section 76f. Between the two, there is a chamfer or blend radius 76g. The geometry allows for the smaller cylinder 76e to fit within the bore of set screw 78. This allows the screwdriver engagement feature 76c to be open and accessible from the top of the assembly. Feature 76c is shown as a pentalobe, but can be any other feature that will engage an instrument tip. The instrument in this case is for counter-torque. The insert can be held steady and the locknut turned by the driving features. This helps prevent the implant from turning as the locking nut is tightened. The groove 76j cut into the larger cylindrical portion 76f also has radii 76j to reduce stress risers. Surface 76f is designed to be pressed on by the bottom surface 78b of locknut 78. The spherical or partially spherical seat 76r is designed to match the spherical location in the lower insert section 74f when the bearing is fully expanded and the upper insert section 76 is in the open position, where lip 76n would be at the top of slot 74g in the lower insert section. Radii 76p and 76g create clearance to fit within the slot radii 74h and 74j. The flat area 76m of the lower insert section aligns with the flat edge of the upper insert section 74s. As with the lower section, a divider 76s or thin web is shown between the spherical seats. Of course, this can be machined away such that the two bearing seats 76r are in direct contact. The dividers 76s and 74s also prevent the rod from being pushed through the bearings and interfering with the other side. Of course, the bearings can have stops within the bearing or locations for the rods to snap into grooves in the bearings, as shown in FIG. 44, bearing 62, thus eliminating the need for dividers.

FIG. 52 shows the upper and lower inserts assembled with the bearings seated within the insert sections. The bearings keep the upper insert section 76 spread apart from lower insert section 72, acting in the manner of a spring. This allows rod 65 to enter the bearing(s). The bearing in this FIGURE has a groove 72e to accept a feature on a rod, such as a snap ring, and a back lip 72f. This lip can be smaller than the rod diameter to prevent the rod from going in too deep, or the same size as the rod to allow the rod to go deeper into the insert.

FIG. 53 shows a variation of the body. Bearing opening 70d is generally rectangular in shape with radii 70g and 70n to minimize the amount of material removed to create the opening, reduce stress risers, and prevent the bearing from contacting a sharp edge. The top of the opening 70f leaves material above it to maintain integrity of the body. With all split designs, forces tend to splay the body outward. This approach eliminates this issue. The threads 70c do not extend through the part, and end before key 70k. The generally smooth bore 70j which has the key machined or formed into it transitions into a tapered section 70m. Between bore 70j and tapered section 70m is a chamfer or radius, or combination of both to allow a smooth transition. At least a portion of the bore 70j above the transition 70n is large enough in diameter to allow the insert taper 74d to expand outward to allow screw head 1c to enter. In this embodiment, body 70 has a larger outside diameter 70p, a smaller diameter section 70q, and a chamfer at the bottom 70r. The larger diameter allows for a larger diameter set screw only in the region needed. Of course, the body can be one diameter or other shapes.

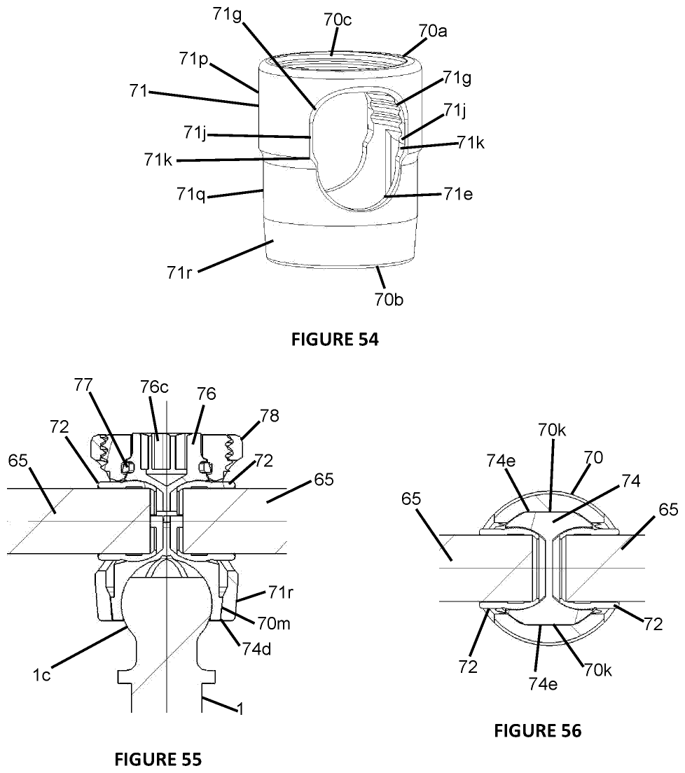

FIG. 54 shows another variation of the body, having a different rod slot shape, which is somewhat key shaped. Body 71 has an upper surface 71a, lower surface 71b, threads 71c, bore 71j, and key 71k. The difference between body and 70 and 71 is the bearing opening. The top of the bearing opening 71j is larger and wider than the lower radiused section 71e. A chamfer and/or radius 71m form a transition between the two openings. When the lock nut is turned, the body is pulled up relative to the insert. As the body is pulled up, the bearing moves into the smaller opening 71e. Thus, regardless of the angle of the bearings perpendicular to the bearing opening, the bearing and rods will be pulled back to straight in the coronal plane. This is extremely beneficial in the treatment of deformity, when curves in the spine need to be corrected.

FIG. 55 is a section view of an assembly in the locked position. As can be seen, taper 70m of the body engages taper 74d of the insert as the lock nut is turned and the body is pulled up relative to the insert. This compresses the insert against screw head 1c while simultaneously locking the bearings and rods in the appropriate positions.

FIG. 56 is a top section view of the assembly cut through the bearings to show the arrangement of the anti-rotation key of insert 74e to body key 70k.

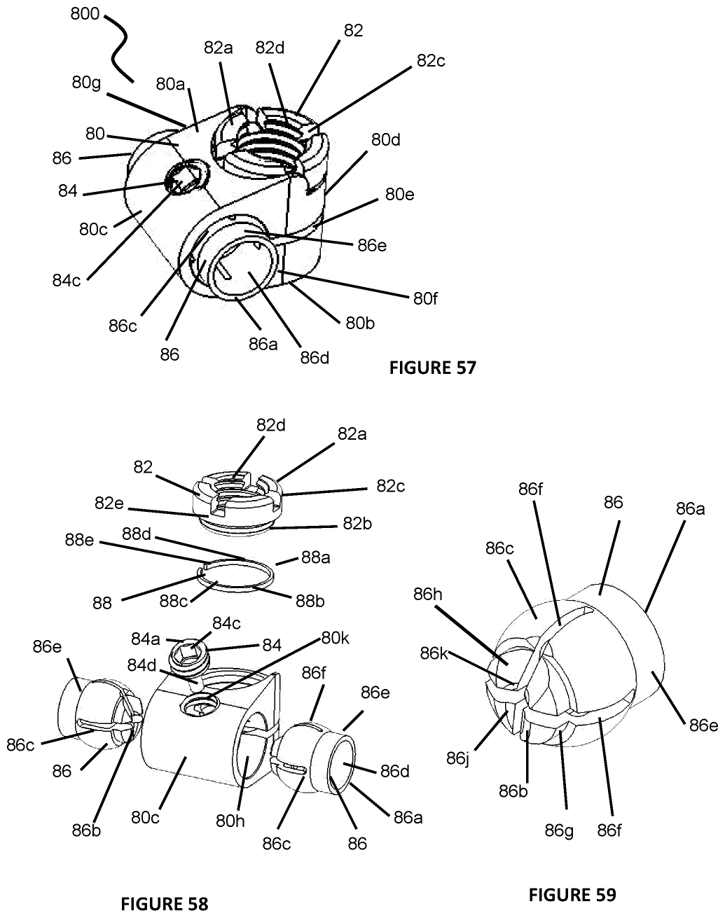

FIG. 57 shows an additional variation represented by general assembly 800. Assembly 800 consists of a main body 80 having a top surface 80a, lower surface 80b, a first face 80c, which is preferably rounded, a second face 80d, two sides 80f and 80g, and a slot 80e. A locking nut 82 consists of a top surface 82a, a feature to engage a driver instrument 82c, and a threaded or at least partially threaded internal bore 82d, and external surface 82e. This locking nut 82 fits within and can be retained within a portion of body 80. A set screw 84 fits within a threaded hole in body 80. Two spherical inserts 86, having a first face 86a, spherical or partially spherical surface 86c, an internal bore 86d for receiving a rod, and an extension 86e are retained within seats in body 80.

The details of general assembly 800 are shown in greater detail in FIG. 58, which shows an assembly view. Body 80 has two seats 80h for accepting two bearings 86. For the end of a construct, there can be a body with only one seat to accept one bearing. The body can also have a threaded hole 80k, which exposes the back of the bearings. Set screw 84 has a top surface 84a, in which is machined or formed a driving feature 84c for attachment to a screwdriver, external threads 84d, and a tip portion 84e. The tip portion has a chamfered tip is designed to engage the back of bearings 86. The locknut 82 lower surface 82b allows for a snap ring 88 to fit within groove 82j in the locknut. The snap ring has an upper surface 88a, lower surface 88b, internal diameter 88c, external diameter 88d, and a slot 88e. The slot 88 allows the snap ring the ability to expand and be compressed into groove 80v in the body to retain the locknut and snap ring assembly. While the snap ring is not required for function and locking, it allows a single assembly for easier surgical use, rather than placing body 80 over the bone screw and then placing the locknut. The seats 80h in body 80 can be spherical, textured, machined with ridges, or have other features to aid in locking the bearings at the desired angle. Having one or more edges, as part of seat 80h, engage the bearing may assist in locking the bearing angle.

Also shown in FIG. 58 and FIG. 59 are the bearings. The bearings have at least one slot 86f, preferably multiple slots, to allow the bearing to be flexible. These slots can run partially through the insert or with one slot extending the full length. Flexibility allows the bearing to enter the body 80 as well as be compressed against rod 92 during tightening of the assembly. The back 86b of bearing 86 has an extended section 86g, which has an upper chamfer 86h, a cylindrical portion 86j, and blend radii 86k. The cylindrical portion 86j allows the tip 84e of set screw 84 to fit tightly within it. As the bearings are moved, the pin tip 84e always sits within a portion of upper chamfer 86h. As the set screw is tightened, the tip pushes the bearing back to a central position. This allows for curvature correction of the spine by use of a simple screwdriver. Depending on the configuration of the set screw and bearing features, coronal, sagittal, or a combination of both can be controlled. Ideally, when the set screw is tightened, the bearings are pulled back to center only in the coronal plane. This allows the deformity to be corrected without creating a flat back issue. Any sagittal correction can be done independently by manipulating the rods up and down with an instrument prior to tightening the locknut. Bearing collar 86 allows for the rod to enter the bearing easier, provide better load distribution to the bearing/rod interface, and reduce stress risers. Preferably at the junction of bearing face 86a and bore 86d, there is a blend radii to prevent a stress riser. Also, having an extended collar allows more room within the bearing for a rod. During correction of a deformity curve, straightening the curve changes the distance between pedicle screws. Thus, there needs to be room for the rods to move within the bearings to take up this change in distance. This will be explained in more detail later.

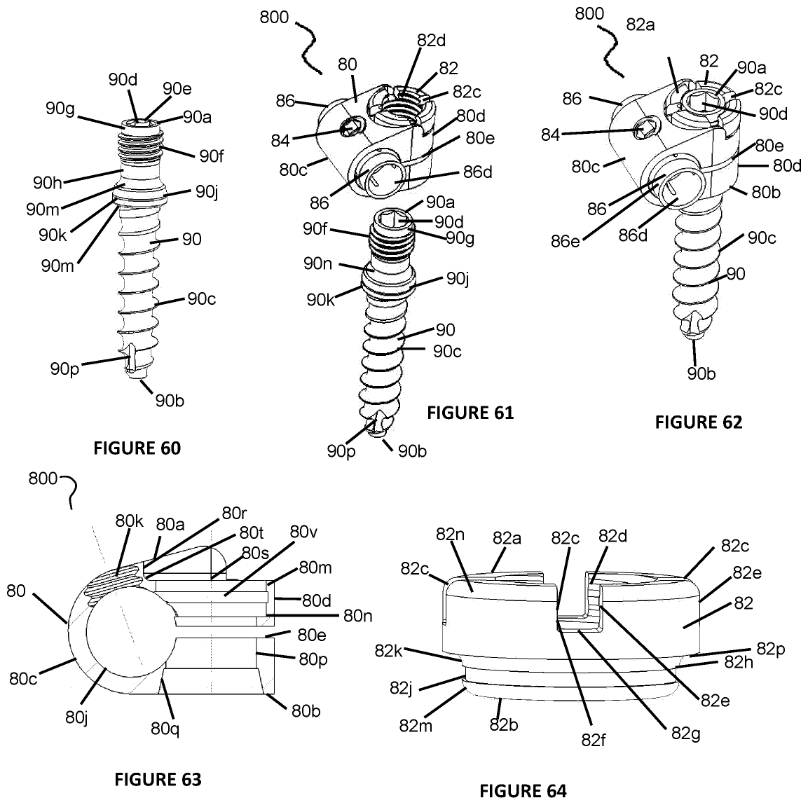

Bone screw 90, as shown in FIG. 60, has an upper surface 90a, tip 90b, a portion with a thread for engaging bone 90c, a driving feature 90d for engaging a screwdriver, and a chamfer 90e to help guide the screwdriver tip into driving feature 90d. The top portion of bone screw 90 has a partially threaded section 90f, an unthreaded section 90g, an unthreaded section 90h below the threads, and a tapered or chamfered section 90j. The chamfered section 90j can also have a cylindrical portion 90k. A blend radius 90m reduced stress and prevents sharp edges. A cutting flute 90p allows the screw to self-tap and form threads into the bone. The screw threads 90c can be single or multiple lead, or any form desired for interfacing cortical and cancellous bone.

FIG. 61 shows the body assembly including body 80, locknut 82, set screw 84, and two bearings 86 in position over bone screw 90. The bone screw is placed in the pedicle first and positioned over the screw head. Once placed on top of the bone screw, the locknut threads 82d engage threads 90f of the bone screw. By tightening locknut 82, body 80 compresses, thus exerting compressive force against the bearings, locking the position of the bearings. Of course, for locking, the rods would be within the bearings. Note that in the case of a single level fusion, whereby two vertebrae are fused together, one rod would span two body/bone screw assemblies, leaving two bearings open in this construct. Thus, rods to not have to be in both bearings for the assembly to lock. In addition, a small section of rod, or a plug can be inserted into the open bearing(s) to keep the bearing(s) free from debris. This would allow for easier addition of more levels, should the fusion need to be extended in the future.

A section view of body 80 is shown in FIG. 63 to better detail the features. The lock nut 82 fits with recess and cylindrical bore 80r, creating a flat bottom 80s for the locknut to press against during locking. This bottom face can be flat or slightly angled, as compression of the locknut to close the body will likely slightly angle the body, as the hinge point is on the opposite side towards 80c. Recess 80v accepts snap ring 88. Through bore 80p extends through the body with clearance to allow screw 90 to enter. The lower internal surface 80q can be cylindrical, but it is preferred to be tapered, so that tightening the locknut will also lock the body securely to the bone screw taper 90j. This helps resist or prevent rotation of the body relative to the bone screw. Splines can also be used on the surface of 80q and/or the bone screw to assist in preventing body to screw rotation. The bearing seats are connected by a central bore or opening 80j. This opening allows the back of the bearings to have clearance to permit full angulation while allowing access to the back of the bearings from the hole 80k. As a note, 80k is shown as threaded. When a set screw is not needed or desired, it can be absent or a simple opening to allow visualization of the rods and bearings.

FIG. 64 shows locknut 82 in more detail. Locknut consists of a larger upper diameter 82e and a smaller diameter section 82h. 82h has a blend radius 82k where it blends from the small section into the larger section, eliminating a sharp transition edge but still leaving a flat lower surface 82p. A groove 82j is machined into section 82h, to accept the snap ring. The slots 82c for connecting locknut 82 with a driver have sidewalls 82e and 82f, with a bottom surface 82g. Of course, the locknut can have other driving features or shapes, such as hexagonal, octagonal, or have other appropriate features that allow for engagement with a nut driver.

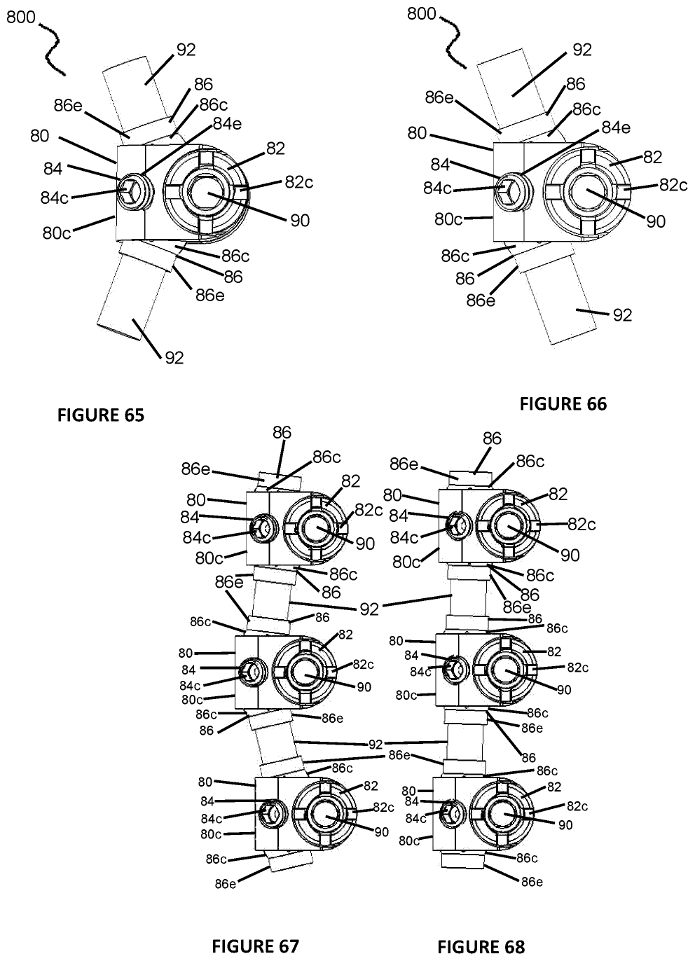

FIGS. 65 and 66 show general assembly 800 engaged on bone screw 90 with rods 92 held within bearings 86. The rod and bearing angle for each side is independent and can angle in any direction, as shown when comparing FIGS. 65 and 66.

The present invention therefore provides for a spinal implant including a body 80 having at least one seat 80h for accepting at least one pivotable bearing 86 for receiving at least one rod 92, the body 80 including a bottom portion for engaging a bone screw 90, and a locking nut 82 received within a top portion of the body 80 and engaging threads 90f on a top portion of the bone screw 90 such that when the locking nut 82 is tightened, the body 80 compresses and locks a position of at least one of the bearings 86.

FIG. 67 shows a combination of three assemblies connected by rods. As can be seen, the two rods are at angles relative to the body. When screws 84 are tightened, the rods straighten, as shown in FIG. 68. Once the rods are straightened, the locknuts can be tightened to secure the assembly. In this example, the end bearings are open and would allow for addition rods and bodies to be added at the time of surgery or later, should they be needed. To add another rod, the locknut is loosened, the new rod inserted, and the locknut retightened. Also, in this example, the rods are the same diameter. It is obvious that the bearings can be machined to accept different diameter rods. Thus, one rod could be 5 mm and another 3.5 mm, such as might be useful in the transition from thoracic to a posterior cervical variation of this system or another posterior cervical system.

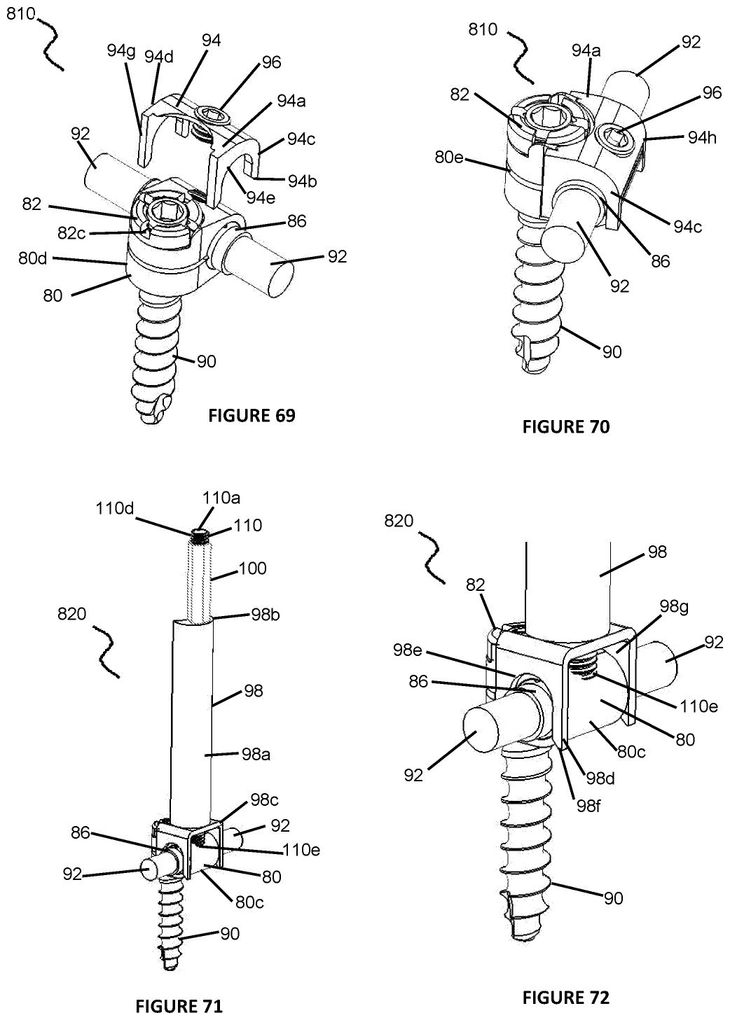

While the focus is on straightening the rods via a central screw, alternatives are shown in FIGS. 69 and 70 and shown as general assembly 810. In FIG. 69, an external clip is used to drive the bearings to a central position. Clip 94 has a top surface 94a, lower surface 94b, sides 94c and 94d, a front edge 94g and back face 94h. The clip is recessed on the inside to fit over body 80, and groove 94e runs through sides 94c and 94d so that they will fit the dimensions of bearing sleeve 86e. Set screw 96 engages the threads in the body, and when tightened, the clip slides over the bearings, pulling them back to straight in at least one plane. FIG. 70 shows the clip in place.

FIGS. 71 and 72 show an instrumentation approach in general assembly 820, whereby an instrument is used to straighten the bearings. A threaded or partially threaded rod 110 engages the threads in body 80 to act as a guide to force instrument 98 into proper alignment. Alignment Instrument 98 has a main shaft 98a, top surface 98b, a U-clip section 98c, back surface 98d, and a cut out 98g to accept body 80. The radiused cut out 98e accepts the bearings, and chamfers 98f help the instrument fit over the bearings when angled and guide them back to straight as the instrument is advanced downward over the bearings. Shaft 110 has a top surface 110a, a threaded portion 110d, and a threaded portion 110e for engaging the implant. A nut 100 engages threads 110. By turning the nut with a handle or nut driver, the instrument end is advanced over the body, driving the bearings back to center. There are other ways of building instrumentation to accomplish this, such as using an instrument to align one bearing at a time rather than both, or different shapes or forms of the instrument.

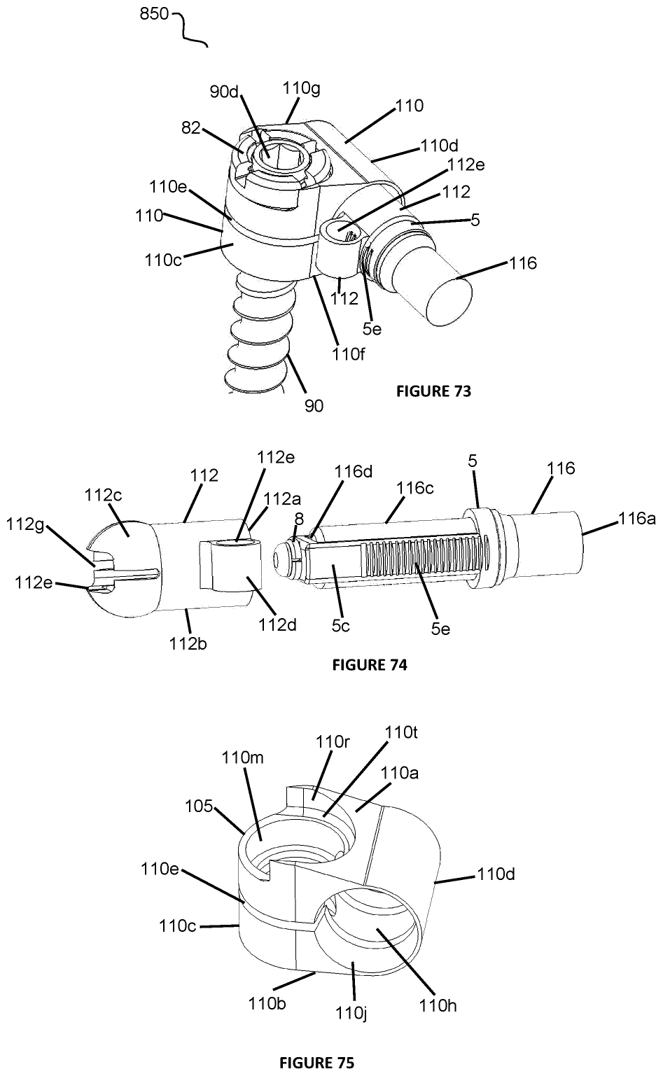

FIGS. 73 through 75 show a combination of features from general assembly 800 and general assembly 100. A rod 116 with rack teeth 5 from general assembly 100 is combined with a bearing designed to accept the geometry of rack 5 and rod 4. The bearing 112 has a cylindrical section 112b with a front face 112a, a spherical bearing portion 112c, an extension 112d with a hole 112e that communicates with the internal bore of bearing 112, a second end 112e, a first slot 112fm and preferably a second and longer slot 112g. As in the previous embodiments, an instrument can be used to engage the rack and move it, in this example through hold 112e. Body 110 is similar to body 80. However, to allow clearance for the rack to move and create compression or distraction, it is preferred to make this a body that has a place for only one bearing—bearing 112. Body 110 has a top surface 110a, lower surface 110b, first face 110c, second face 110d, a spherical seat for the spherical bearing 112c, and chamfer 110j to allow clearance for the rack assembly to angulate. A recess 110r is formed for the locknut to fit within, which leaves ledge 110s for the nut to press against during locking. The internal bore 110m accepts portion 82h of the locknut and can be adapted to work with or without a snap ring. Rod 116 is a cylindrical rod 116a, with a cylindrical rod portion 116c, and flats 116d.

The advantages of the implant shown by general assembly 800, 810, and 820, are significant in the ability to allow for angulation of at least one rod without the need for a polyaxial screw interface. By eliminating a spherical screw head, the height of the assembly is extremely low, allowing for easier placement, and for pediatric deformity, reducing soft tissue issues often a problem with normal polyaxial screws. In addition, screw 90 is always accessible through the locknut, allowing the screw to be moved up and down in the pedicle as needed to adjust assembly height. With two bearings adjustable within a large range and the ability to adjust assembly height, the surgeon has new tools at his disposal to treat complex pediatric deformity.

In the treatment of pediatric deformity, there are generally two ways to correct spinal curvature. Once technique, called derotation, involves bending a long rod into an approximate proper spinal curve, connecting it to spinal implants, and rotating the rod 90 degrees, thereby acting as a cam to force the spine into alignment. This is effective. However, there are issues. First, it is challenging and requires a long incision. Secondly, it induces high amounts of stress on the body during derotation. Third, the instrumentation to accomplish the task is expensive and is not designed to do partial adjustments. Fourth, the surgeon has to commit to longer constructs then might be necessary, thereby fusing vertebrae that might not need to be fused.

With the implants shown in this disclosure, the ability to add rods one at a time and add in levels or vertebrae as needed changes the treatment option. This allows the surgeon to treat the apex of the curve and straighten it to see what levels actually need to be fused. Additional levels can be added one at a time until the desired result is achieved. In addition, with a two bearing variation, one bearing can be left open to provide a simple attachment mechanism to add more implants to at a later surgery. This is often challenging with other implant systems.

Furthermore, the ability to adjust angulation of the rods via a central access port is novel and provides further advantages. By adjusting the central set screw with a simple tool, the spine can be straightened in at least one plane, eliminating complex instrumentation and possibly providing a better result in a more incremental and gentle manner. Rather than adjust all levels at one, each implant can be adjusted one at a time, gently pulling the spine back into position.

As the angulation is adjusted and the spine straightened, the distance from screw post to screw post center also changes. Therefore, it is important to allow sliding of the rod within the bearings during the angulation adjustment. Thus, there are multiple rod lengths to pick from. A trial system can be used to determine proper length of the rod for the procedure. Once the locknut is tightened, the body squeezes closed, and the bearings are compressed on the rods, which prevents any sliding of the rods. Compression and distraction can also be done prior to locking the locknut.

For purposes of design and manufacturing, surface finish and material effect the implant strength and testing results. Ti-6Al-4V ELI bone screws with a machined tooth pattern on the head works well. Other titaniums and titanium alloys can also be used. While stainless steel and other materials, such as polymers, can be used, they are less preferred. There are different grades of titanium, and different alloys, each may have a different effect and require different surface or no surface treatment. The alloys chosen were based on their standard use in the industry, biocompatibility, and properties.

Surface roughness on the screw head and/or screw head seat can be applied in a number of ways, including machining, chemical etching, grit blasting, or other means. It is preferable to provide a machined feature on the screw head which creates small grooves around the surface. While this can be done as individual circular grooves, the machined pattern can be run in a helix, effectively creating a shallow thread over the spherical surface. It is preferable to create the surface over the entire spherical area to be certain that rotation of the bone screw head in the insert or collet seat still maintains engagement of the surface roughness pattern regardless of the angulation.

As the connector assemblies in the different forms described are self-contained and small, it is possible to supply the polyaxial screw/rack/rod construct assembly complete, sterile packed, and provide a range of sterile sizes ready for surgery. As can be imagined, this implant system also requires a minimal number of instruments, simplifying the surgical implant procedure and allowing the instruments to be supplied sterile.

The present invention provides for a spinal implant assembly including a first spinal implant having at least one side opening and at least one bearing for receiving a spinal rod to a specific or maximum depth, a second spinal implant having at least one side opening and at least one bearing for receiving a spinal rod to a specific or maximum depth, and a rod that can be inserted through the side opening and bearing of the first spinal implant and connected to the second spinal implant by inserting the rod through the side opening and into the bearing of the second spinal implant.

The present invention also provides for a spinal implant assembly including a first spinal implant having two bearings for receiving spinal rods and a second spinal implant having at least one bearing for receiving a spinal rod such that when the first spinal implant and the second spinal implant are implanted, at least one bearing remains open for adding on an additional rod and spinal implant.

The present invention also generally provides for a method of using the spinal implant described above by inserting the spinal implant at a bone structure of a spine, tightening the locking nut, drawing the body upwards, compressing the insert against the bone screw head, and contacting a lower surface of the locking nut and the rod assembly and compressing the rod assembly against the insert, thus locking the rod assembly and bone screw. This method can further include steps that have previously been described above. For example, the method can include partially locking the body by turning the locking nut at a torque less than that needed to fully lock the body. The method can further include the step of applying a counter torque by engaging a counter torque shaft with a feature for accepting an instrument tip on the insert. The method can further include the step of repositioning the spinal implant by loosening the locking nut, forcing the body downwards, retracting the insert, and removing the body and insert and repositioning the body and insert. The method can further include the step of repositioning the rod assembly. The method can further include the step of actuating a rack section of the rod assembly and moving the spine. The method can be used to treat lumbar or thoracic spine. The method can further include before the inserting step, the step of implanting a bone screw in the bone structure, and snapping the insert over a top of the bone screw. The method can further include the step of connecting at least a second spinal implant to the spinal implant through the rod assembly. The connecting step can be performed by connecting the rod assembly through a spherical bearing within a body of a second spinal implant. The method can further include the step of compressing the bearings with the locking nut. The method can further include the step of driving bearings to center. The connecting at least a second spinal implant can be performed at a later time, such as at a later surgery when needed. The compression step can be performed by turning an instrument and providing linear motion. Each of these steps are further described above.

The present invention also provides for a method of using the spinal implant shown in FIG. 60. This method can be performed by inserting a spinal implant at a bone structure of a spine including a body including two seats having pivotable bearings receiving at least one rod, the body including a bottom portion for engaging a bone screw, and a locking nut received within a top portion of the body and engaging threads on a top portion of the bone screw, tightening the locking nut, compressing the body and thereby exerting force against the bearings, and locking the position of the bearings. This method can also include steps described above.

The present invention also provides generally for a method of aligning and locking a spinal implant, by turning a locking nut, pulling a body of the spinal implant upwards and aligning an insert within the body, and locking the spinal implant. Each of these steps are described in detail above.

The foregoing description and accompanying drawings illustrate the principles, exemplary embodiments, and modes of operation of the invention. However, the invention should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the invention as defined by the following claims.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A spinal implant, comprising a body having an insert received within a portion of said body, said insert receiving a bone screw at a bottom portion for attachment to a spine, a rod assembly received within a rod slot on a side portion of said body wherein said rod slot does not extend through a top surface of said body, and a locking nut received within a top portion of said body and interlocking with a top portion of said insert.

2. The spinal implant of claim 1, wherein said rod assembly includes an end for connection with a second spinal implant in a shape chosen from the group consisting of cylindrical and spherical.

3. (canceled)

4. (canceled)

5. The spinal implant of claim 1, wherein said insert includes an opening that matches a shape of an outside of said rack member.

6. The spinal implant of claim 1, wherein said insert includes a rod seat for a rod to sit and slide within.

7. (canceled)

8. The spinal implant of claim 1, wherein said body includes an instrument opening that allows access to a gear rack.

9. (canceled)

10. The spinal implant of claim 1, wherein said rod assembly includes a rod, rack section, and snap ring for maintaining said rack section on said rod, wherein said rack section freely turns around a cylindrical section of said rod.

11. (canceled)

12. (canceled)

13. The spinal implant of claim 1, wherein said insert includes a groove at a top section for receiving a snap ring, and said locking nut includes a groove for receiving said snap ring, whereby said locking nut can freely turn around said insert top section but is axially constrained, and said body can move up or down relative to said insert by turning said locking nut.

14. The spinal implant of claim 10, wherein said locking nut compresses said rod of said rod assembly.

15. The spinal implant of claim 1, wherein multiple spinal implants are interconnected through at least one rod assembly.

16. (canceled)

17. The spinal implant of claim 16, wherein said cylindrical section includes a feature for retaining a collet that expands and contracts to accept said bone screw.

18. (canceled)

19. (canceled)

20. (canceled)

21. The spinal implant of claim 1, wherein said body further includes at least one spherical bearing for accepting a rod assembly.

22. The spinal implant of claim 21, wherein said rod assembly includes an engaging mechanism for engaging said spherical bearing.

23. The spinal implant of claim 21, wherein said spherical bearing accepts said rod assembly at varying angles.

24. The spinal implant of claim 12, wherein said rod assembly is slideable within said spherical bearing.

25. The spinal implant of claim 21, wherein said body includes a rectangular bearing opening for receiving said spherical bearing.

26. The spinal implant of claim 21, wherein said spherical bearing is flexible.

27. (canceled)

28. (canceled)

29. (canceled)

30. The spinal implant of claim 1, wherein said body further includes a set screw or locknut that when tightened, pulls said spherical bearings to center.

31. (canceled)

32. (canceled)

33. (canceled)

34. (canceled)

35. (canceled)

36. The spinal implant of claim 34, wherein said body further includes a set screw or locking nut that when tightened, pulls said spherical bearings towards the center in at least one plane.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A spinal implant assembly comprising a first spinal implant having at least one side opening and at least one bearing for receiving a spinal rod to a specific or maximum depth, a second spinal implant having at least one side opening and at least one bearing for receiving a spinal rod to a specific or maximum depth, and a rod that can be inserted through said side opening and said bearing of said first spinal implant and connected to said second spinal implant by inserting said rod through said side opening and into said bearing of said second spinal implant.

56. A spinal implant assembly comprising a first spinal implant having two bearings for receiving spinal rods and a second spinal implant having at least one bearing for receiving a spinal rod such that when said first spinal implant and said second spinal implant are implanted in an individual, at least one bearing remains open for adding on an additional rod and spinal implant.
